(19) United States
(12) Patent Application Publication
(10) Pub. No.: US 2008/0138904 A1
(43) Pub. Date: Jun. 12, 2008

(54) Method Of Breeding Cells To Improve Tolerance To Short Chain Fatty Acids

(76) Inventors: Shigeru Nakano, Aichi (JP); Masahiro Fukaya, Aichi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/793,145

(22) PCT Filed: Dec. 12, 2005
(86) PCT No.: PCT/JP05/22742

§ 371 Date: Jun. 14, 2007

(30) Foreign Application Priority Data
Dec. 17, 2004 (JP) 2004-365521
Jan. 24, 2005 (JP) 2005-014984

(51) Int. Cl. C12N 15/09; C12N 1/21

(52) U.S. Cl. 435/471; 435/252.31; 435/252.33

(57) ABSTRACT

It is intended to provide a method of conferring to microbial cells or the like, tolerance to a short chain fatty acid, including formic acid and acetic acid, which is harmful to the growth thereof without suppressing the growth of the cells while maintaining a high productivity of useful substances. It is also intended to provide a means for efficiently culturing a microorganism in the presence of a short chain fatty acid, or a means for producing a useful short chain fatty acid via fermentation.

The present invention provides a method of breeding cells to improve tolerance to a short chain fatty acid, wherein a gene encoding a protein having a function of transporting a short chain fatty acid from the inside of cells to the outside of cells, is transformed into and expressed in the cells; a high cell-density culture method for producing useful substances using the cell; and a method of preparing a fermentation broth comprising a short chain fatty acid, wherein the cells are cultured under the conditions such that the cells produce a short chain fatty acid.

### TECHNICAL FIELD

The present invention relates to a method of breeding cells to improve tolerance to a short chain fatty acid, and in particular, to a method of breeding cells to improve tolerance to a short chain fatty acid by introducing a gene conferring tolerance to a short chain fatty acid into cells of a microorganism or the like, and expressing the gene therein, and as for the use of such microbial cells obtained by the method, to a method for high cell-density culture for producing useful substances, and efficient preparation of a fermentation broth comprising a short chain fatty acid.

### BACKGROUND ART

It is conventional to add, in industrial culture of microorganisms, carbon sources such as glucose and the like, nitrogen sources such as peptone and the like, inorganic substances such as sulfates, phosphates, sodium and the like, trace elements such as calcium, zinc and the like, and growth factors such as purine, pyrimidine and the like, as nutrients to the culture medium.

However, there has been a problem that when these nutrients are consumed upon growth of the microorganisms, the microorganisms produce in the medium short chain fatty acids, such as formic acid, acetic acid and the like, which are toxic to the growth of the microorganisms, and as a result, the growth of the microorganisms ceases.

In particular, Escherichia coli, which is frequently used for the production of recombinant proteins, is widely used as a host bacterium for high cell-density under aerobic conditions. In the high cell-density aerobic culture of E. coli, it is conventional to add an appropriate amount of glucose to the growth medium, but this causes to accumulation of short chain fatty acids mainly comprising acetic acid in the culture. These short chain fatty acids are main factors that inhibit high cell-density culture, and they also inhibit the production of recombinant proteins (see, for example, Non-Patent Document 1 and Non-Patent Document 2).

To address this problem, a fed-batch culture method in which nutrients are fed continuously or intermittently as the culture proceeds to the medium during culture (see, for example, Non-Patent Document 3), a dialysis culture method in which extracellular products are removed to the outside of the culture system using a dialysis membrane or the like (see, for example, Non-Patent Document 4), and others are employed for the culture of microorganisms.

By the fed-batch culture method, it is possible to arbitrarily control the concentration of a specific component in the medium, and for example, it is possible to maintain a sugar concentration in the medium within the optimal range for the cultured microorganism. Consequently, a desired microorganism can be efficiently cultured, and this method is widely employed. However, even by use of the fed-batch culture method, the production of organic acids cannot be suppressed, and there still remains several problems such as repression of growth rate, reduction in yield of recombinant proteins, and the like which are caused by the produced organic acids.

Furthermore, although the dialysis culture method allows the reduction of the influence of the produced short chain fatty acids by eliminating extracellular products, it also has problems such that specialized equipments are needed and the process is complicated.

In addition, in the glucose metabolism of E. Coli under aerobic condition, when carbon sources are added in an amount exceeding the metabolizing capability of the TCA cycle, acetic acid is produced and released to the outside of the cells, and thereby cell growth and production of recombinant proteins are inhibited (see, for example, Non-Patent Document 5).

Since acetic acid is biosynthesized from acetyl-CoA by phosphotransacetylase (PTA) and acetic acid kinase (ACK), Non-Patent Document 5 describes the preparation of E. coli mutants that are deficient in the genes of these enzymes (PTA and ACK). However, in these mutants, although the biosynthetic pathway for acetic acid (PTA and ACK) is inactivated, still there are other pathways for the production of acetic acid. Thus, the amount of acetic acid produced was lowered, but the production was not completely repressed. Furthermore, the defective mutants excessively accumulated organic acids other than acetic acid, such as lactic acid, pyruvic acid and the like (see, for example, Non-Patent Document 6 and Non-Patent Document 7).

As such, development of microorganisms which are incapable of producing short chain fatty acids, such as formic acid, acetic acid and the like, which are produced during culture and are harmful to cell growth, has not yet been succeeded.

Meanwhile, in industrial production of these short chain fatty acids by microbial fermentation, development of an efficient method for producing the short chain fatty acids on a large scale is required. In this case, microorganisms showing sufficient tolerance to a short chain fatty acid would be needed, but such microorganisms have not been developed.

Meanwhile, a number of acetic acid bacterium-derived genes having a function of improving tolerance to acetic acid have been found by the present inventors, and among these genes included is a gene cluster comprising a motif of an ATP-binding cassette (see, for example, Patent Document 1).

These genes having the motif of an ATP-binding cassette are generically referred to as the ABC transporter family, and are assumed to encode proteins which serve as transporters having a function of transporting metabolites, drugs and the like from the inside of the cells to the outside of the cells, or from the outside of cells to the inside of cells.

When one of these genes constituting the ABC transporter family (ABC transporter gene) was ligated to a multicopy number vector and introduced into acetic acid bacterium, the resulting transformant (acetic acid bacterium) showed improved tolerance to acetic acid, but there was no influence on the tolerance to other organic acids (see, for example, Japanese Unexamined Patent Application Publication No. 2003-289868).

Patent Document 1: JP-A 2003-289868

Non-Patent Document 1: Appl. Environ. Microbiol., Vol. 56, p. 1004-1011 (1990)
Non-Patent Document 2: Biotechnol. Bioeng., Vol. 39, p. 663-671 (1992)

### Non-Patent Document 3: Trends Biotechnol., Vol. 14, p. 98-100 (1996)

Non-Patent Document 4: Appl. Microbiol. Biotechnol., Vol. 48, p. 597-601 (1997)
Non-Patent Document 5: Biotechnol. Bioeng., Vol. 35, p.732-738 (1990)
Non-Patent Document 6: Biotechnol. Bioeng., Vol. 38, p. 1318-1324 (1991)
Non-Patent Document 7: Biosci. Biotechnol. Biochem., Vol. 58, p. 2232-2235 (1994)

### DISCLOSURE OF THE INVENTION

### Problem to be Solved by the Invention

Therefore, firstly, it is an object of the present invention to provide a method of conferring to microbial cells or the like tolerance to short chain fatty acids including formic acid and acetic acid which are harmful to the growth thereof, without suppressing the growth of the cells and with maintaining a high productivity of a useful substances.

Secondly, it is another object of the invention to provide a means for efficient culture of a microorganism in the presence of a short chain fatty acid, and a means for producing a useful short chain fatty acid via fermentation, with regard to the use of above-mentioned microorganism.

### Means for Solving the Problem

While conducting the study on a means to solve the above-described problems, the inventors of the present invention focused their attention on acetic acid, which is one of the short chain fatty acids, and took notice of an acetic acid bacterium-derived gene cluster which is assumed to belong to an ABC transporter family possessed by acetic acid bacteria, which confer high tolerance to acetic acid and is used for acetic acid fermentation in the manufacture of vinegar. And, the inventors extensively investigated as to whether the ABC transporter gene constituting the ABC transporter family can be introduced into and expressed in heterogeneous cells other than the original cells of acetic acid bacteria.

As a result, they discovered that transformants were also obtained from Escherichia coli, which belongs to a genus completely different from acetic acid bacteria and inherently possesses low ability to oxidize alcohol, or Gluconacetobacter diazotrophicus, which belongs to acetic acid bacteria but has low ability of acetic acid fermentation. They also unexpectedly discovered that, unlike acetic acid bacteria, the growth of transformants of E. coli or G. diazotrophicus was not repressed by a short chain fatty acid (having 5 or fewer carbon atoms) other than acetic acid, that is, these bacteria showed tolerance to a short chain fatty acid. Furthermore, it was confirmed that introduction of the gene did not cause reduction in productivity of the useful substances whose productivity was the same as that of the original strain, or in productivity of recombinant proteins conferred by exogenously introduced gene.

Further, the inventors studied on the mechanism how the ABC transporter gene enhances tolerance to short chain fatty acid using transformants of acetic acid bacteria. Based on the finding that the intracellular acetic acid concentration of the transformant was reduced compared to that of the original strain, they confirmed that the transformant acquired an ability of transporting acetic acid from the inside of the cells to the outside of the cells, that is, to the medium. Therefore, it was assumed that this function could be applied as a mechanism for improving the tolerance and applied not only to acetic acid bacteria, but also to heterogeneous cells.

And, when a microorganism transformed with the gene was cultured at high cell-density, it can sufficiently grow even in the presence of a short chain fatty acid compared with a microorganism that was not transformed. This also leads to the finding that the transformant is useful for industrial culture.

Furthermore, it was also discovered that the concentration of an accumulated short chain fatty acid such as propionic acid and the like was increased.

The present invention is based on the above-stated findings.

Thus, the invention comprises the following (1) to (11).

(1) A method of breeding cells to improve tolerance to a short chain fatty acid, wherein a gene encoding a protein having a function of transporting a short chain fatty acid from the inside of cells to the outside of cells, is introduced and expressed in the cells.

(2) The method of breeding cells according to (1), wherein the gene encoding the protein having a function of transporting a short chain fatty acid from the inside of cells to the outside of cells is a DNA represented by the following (a) or (b):

(a) a DNA that comprises a nucleotide sequence consisting of nucleotide numbers 301 to 2073 in the nucleotide sequence set forth in SEQ ID NO: 1 of the Sequence Listing; or

(b) a DNA that comprises a nucleotide sequence which hybridizes with a probe comprising a nucleotide sequence consisting of nucleotide numbers 301 to 2073 in the nucleotide sequence set forth in SEQ ID NO: 1 of the Sequence Listing or comprising at least a part of said nucleotide sequence under stringent conditions, and that encodes a protein which is capable of enhancing tolerance to acetic acid.

(3) The method of breeding cells according to (1), wherein the gene encoding the protein having a function of transporting a short chain fatty acid from the inside of cells to the outside of cells is a DNA represented by the following (a) or (b):

(a) a DNA that comprises a nucleotide sequence consisting of nucleotide numbers 331 to 2154 in the nucleotide sequence set forth in SEQ ID NO: 3 of the Sequence Listing; or (b) a DNA that comprises a nucleotide sequence which hybridizes with a probe comprising a nucleotide sequence consisting of nucleotide numbers 331 to 2154 in the nucleotide sequence set forth in SEQ ID NO: 3 of the Sequence Listing or comprising at least a part of said nucleotide sequence under stringent conditions, and that encodes a protein which is capable of enhancing tolerance to acetic acid.

(4) The method of breeding cells according to (1), wherein the gene encoding the protein having a function of transporting a short chain fatty acid from the inside of cells to the outside of cells is a DNA represented by the following (a), (b), (c) or (d):

(a) a DNA that comprises a nucleotide sequence consisting of nucleotide numbers 1724 to 2500 and a nucleotide sequence consisting of nucleotide numbers 1724 to 2500 in the nucleotide sequence set forth in SEQ ID NO:5 of in the Sequence Listing;

(b) a DNA that comprises both a nucleotide sequence which hybridizes with a probe comprising a nucleotide sequence consisting of nucleotide numbers 1002 to 1724 in the nucleotide sequence set forth in SEQ ID NO:5 of the Sequence Listing or comprising at least a part thereof under stringent conditions, and a nucleotide sequence consisting of nucleotide numbers 1724 to 2500 in the nucleotide sequence set forth in SEQ ID NO:5 of the Sequence Listing, and that encodes a protein complex which is capable of enhancing tolerance to acetic acid;

(c) a DNA that comprises both a nucleotide sequence consisting of nucleotide numbers 1002 to 1724 in the nucleotide sequence set forth in SEQ ID NO:5 of the Sequence Listing, and a nucleotide sequence which hybridizes with a probe comprising a nucleotide sequence consisting of nucleotide numbers 1724 to 2500 in the nucleotide sequence set forth in SEQ ID NO:5 of the Sequence Listing or comprising at least a part thereof under stringent conditions, and that encodes a protein complex which is capable of enhancing tolerance to acetic acid; or

(d) a DNA that comprises both a nucleotide sequence which hybridizes with a probe comprising a nucleotide sequence consisting of nucleotide numbers 1002 to 1724 in the nucleotide sequence set forth in SEQ ID NO:5 of the Sequence Listing or comprising at least a part thereof under stringent conditions, and a nucleotide sequence which hybridizes with a probe comprising a nucleotide sequence consisting of nucleotide numbers 1724 to 2500 in the nucleotide sequence set forth in SEQ ID NO:5 of the Sequence Listing or comprising at least a part thereof under stringent conditions, and that encodes a protein complex which is capable of enhancing tolerance to acetic acid.

(5) The method of breeding cells according to (1), wherein the gene encoding the protein having a function of transporting a short chain fatty acid from the inside of cells to the outside of cells is a DNA represented by the following (a) or (b):

(a) a DNA that comprises a nucleotide sequence consisting of nucleotide numbers 249 to 1025 in the nucleotide sequence set forth in SEQ ID NO: 8 of the Sequence Listing; or

(b) a DNA that comprises a nucleotide sequence which hybridizes with a probe comprising a nucleotide sequence consisting of nucleotide numbers 249 to 1025 in the nucleotide sequence set forth in SEQ ID NO: 8 of the Sequence Listing or comprising at least a part of said nucleotide sequence under stringent conditions, and that encodes a protein which is capable of enhancing tolerance to acetic acid.

(6) The method of breeding cells according to any one of (1) to (5), wherein the short chain fatty acid is formic acid, acetic acid, propionic acid, butyric acid, isobutyric acid, or valeric acid.

(7) Cells bred by the method according to any one of (1) to (5), wherein the cells are microbial cells.

(8) The cells according to (7), wherein the microbial cells are cells of acetic acid bacteria, bacteria belonging to the genus Escherichia, or genus Bacillus.

(9) A method for high cell-density culture that uses the cells according to (8).

(10) The method for high cell-density culture according to (9), wherein the cells are cultured in the presence of a short chain fatty acid.

(11) A method of preparing a fermentation broth comprising a short chain fatty acid, wherein the cells according to (8) are cultured under the conditions such that the cells produce a short chain fatty acid.

### Effect of the Invention

According to the present invention, cells that are conferred tolerance to a short chain fatty acid and show improved tolerance to a short chain fatty acid can be bred. Furthermore, when the method of breeding of the invention is applied to microbial cells, cells whose growth is not affected by short chain fatty acids that are produced during culture and harmful to cell growth, can be efficiently obtained.

The microbial cells exhibiting tolerance to a short chain fatty acid obtained by the invention can also be applied to high cell-density culture in which short chain fatty acids are produced. Also, the cells can be used for the preparation of fermentation broth comprising short chain fatty acids at high concentrations. Particularly in the case of Escherichia coli to whose tolerance to a short chain fatty acid is conferred, or the like, the bacterial cells show significantly improved ability to grow in medium and can efficiently accumulate a high concentration of short chain fatty acids, thus being industrially useful.

### BRIEF DESCRIPTION OF THE DRAWINGS

FIG. 1 is a schematic diagram showing a construction of E. coli-Acetobacter shuttle vector pGI18.

FIG. 2 is a set of graphs showing time courses in growth of the transformant and non-transformed strain according to Example 1 in (a) a medium with no addition, (b) a medium added with formic acid, (c) a medium added with acetic acid, and (d) a medium added with propionic acid.

FIG. 3 is a set of graphs showing time courses in growth of the transformant and non-transformed strain according to Example 1 in (a) a medium added with butyric acid, (b) a medium added with isobutyric acid, and (c) a medium added with n-valeric acid.

FIG. 4 is a set of graphs showing time courses in growth of the transformant and non-transformed strain according to Example 2 in (a) a medium added with formic acid, and (b) a medium added with acetic acid.

FIG. 5 is a schematic diagram showing a restriction enzyme map of a Gluconacetobacter entanii-derived gene fragment, the location of the gene conferring tolerance to short chain fatty acids, and the DNA fragment inserted into plasmid pABC31.

FIG. 6 is a set of graphs showing time courses in growth of the transformant and non-transformed strain according to Example 3 in (a) a medium added with formic acid, and (b) a medium added with acetic acid.

FIG. 7 is a set of graphs showing time courses in growth of the transformant and non-transformed strain according to Example 4 in (a) a medium added with formic acid, and (b) a medium added with acetic acid.

FIG. 8 is a schematic diagram showing a restriction enzyme map of a cloned Gluconacetobacter entanii-derived gene fragment, the location of the gene conferring tolerance to short chain fatty acids, and the DNA fragment inserted into plasmid pABC41.

FIG. 9 is a set of graphs showing time courses in growth of the respective cells according to Example 6(1), and time courses in amounts of total organic acids and acetic acid in the broth.

FIG. 10 is a graph showing time courses in growth in glucose fed-batch culture according to Example 6(2).

FIG. 11 is a graph showing time courses in growth of the transformant and non-transformed strain according to Example 7 in a medium comprising acetic acid.

FIG. 12 is a schematic diagram showing a restriction enzyme map of a DNA comprising a nucleotide sequence set forth in SEQ ID NO: 1 of the Sequence Listing, the location of the gene conferring tolerance to a short chain fatty acid, and the DNA fragment inserted into plasmid pABC1.

### BEST EMBODIMENT FOR CARRYING OUT THE INVENTION

Hereinafter, the present invention will be described in detail.

An object of the invention is to provide a method of breeding cells which are transformed with a gene having a function of transporting a short chain fatty acid from the inside of cells to the outside of cells (gene conferring tolerance to short chain fatty acids), cells improved by the method of breeding, and a method for high cell-density culture using said cells, and a method of preparing a fermentation broth comprising a useful short chain fatty acid.

[1] Method of Breeding of the Invention

The method of breeding cells to improve tolerance to a short chain fatty acid of the invention is characterized in that, as described in claim 1, a gene encoding a protein having a function of transporting a short chain fatty acid from the inside of cells to the outside of cells (gene conferring tolerance to short chain fatty acids) is introduced into and expressed in the cells.

Here, “protein having a function of transporting a short chain fatty acid from the inside of cells to the outside of cells” means a protein which exhibits, in cells of the present invention, a function of transporting a short chain fatty acid which is incorporated into the cells or produced by a intracellular activity such as metabolism or the like, to the outside of cells. For example, the protein means a protein that can reduce the intracellular acetic acid concentration by 15 to 20% or more compared to that of the parental strain, in a medium where acetic acid is added at the concentration affecting cell growth. Specific examples of such protein include a protein having an amino acid sequence set forth in SEQ ID NO: 2, 4 or 9 of the Sequence Listing, and protein complexes having amino acid sequences set forth in SEQ ID NOs.: 6 and 7.

Furthermore, proteins comprising mutations such as substitution, deletion, insertion, addition, inversion and the like of one or multiple, preferably one or a few, amino acids in the respective amino acid sequences set forth in SEQ ID NOs: 2, 4 and 9 of the Sequence Listing, are also included in the proteins mentioned above, as long as they have the function of transporting a short chain fatty acid from the inside of cells to the outside of cells. Protein complexes comprising mutations such as substitution, deletion, insertion, addition, inversion and the like of one or multiple, preferably one or a few, amino acids in any of the amino acid sequences set forth in SEQ ID NO: 6 and/or 7, are likewise included in the proteins mentioned above, as long as they have the function of transporting a short chain fatty acid from the inside of cells to the outside of cells.

Furthermore, the gene encoding the aforementioned protein (gene conferring tolerance to short chain fatty acids) means a gene which contains a coding region for the protein, and can be transformed into and expressed in cells. Representative examples of such genes include an acetic acid bacterium-derived ABC transporter genes forming a cluster which is assumed to belong to an ABC transporter family. An ABC transporter gene means a gene comprising a motif of an ATP-binding cassette, or a gene forming an operon with a gene comprising the motif which encodes a protein to form a protein complex. The gene comprising a motif of an ATP-binding cassette is generically referred to as the ABC transporter family, and is assumed to encode proteins serving as transporters, which have a function of transporting metabolites, drugs and the like from the inside of cells to the outside of cells, or from the outside of cells to the inside of cells.

Examples of such ABC transporter genes include a gene comprising a coding region encoding a protein having an amino acid sequence set forth in SEQ ID NO: 2, 4, 6, 7 or 9 of the Sequence Listing and specifically include DNAs comprising a nucleotide sequence consisting of nucleotide numbers 301 to 2073 of SEQ ID NO: 1, a nucleotide sequence consisting of nucleotide numbers 331 to 2154 of SEQ ID NO: 3, a nucleotide sequence consisting of nucleotide numbers 1002 to 1724 and a nucleotide sequence consisting of nucleotide numbers 1724 to 2500 of SEQ ID NO: 5, or a nucleotide sequence consisting of nucleotide numbers 249 to 1025 of SEQ ID NO: 8, in the nucleotide sequence set forth in SEQ ID NO: 1, 3, 5 or 8, as described in (a) of claims 2 to 5.

These DNAs may be ones that contain the specific nucleotide sequences, and it is also possible to use, for example, DNAs consisting of the full length of each of the nucleotide sequences set forth in SEQ ID NOs: 1, 3, 5 and 8 of the Sequence Listing.

These DNAs can be easily obtained by PCR using DNAs which are designed based on a nucleotide sequence set forth in SEQ ID NO: 1, 3, 5 or 8 as the primers and DNA of acetic acid bacteria (an acetic acid bacteria of genus Acetobacter or genus Gluconacetobacter) as the template, respectively. In particular, the DNAs consisting of the respective nucleotide sequences set forth in SEQ ID NOs: 1 and 3 of the Sequence Listing can be obtained from Acetobacter aceti strain No. 1023 (deposited at International Patent Organism Depositary, National Institute of Advanced Industrial Science and Technology (Central 6, 1-1, Higashi 1-chome, Tsukuba-shi, Ibaraki-ken, Japan) (former designation: Fermentation Research Institute Agency of Industrial Science and Technology, the Ministry of International Trade and Industry, former address: 1-3, Higashi 1-chome, Tsukuba-shi, Ibaraki-ken, Japan) under accession number FERM BP-2287 on Jun. 27, 1983 (transferred from the original deposit on Feb. 13, 1989)) as the template. Furthermore, the DNAs consisting of the respective nucleotide sequences set forth in SEQ ID NOs: 5 and 8 of the Sequence Listing can be respectively obtained from one species of Gluconacetobacter entanii, Acetobacter altoacetigenes strain MH-24 (deposited at International Patent Organism Depositary, National Institute of Advanced Industrial Science and Technology (Central 6, 1-1, Higashi 1-chome, Tsukuba-shi, Ibaraki-ken, Japan) (former title: Fermentation Research Institute Agency of Industrial Science and Technology, Ministry of International Trade and Industry, former address: 1-3, Higashi 1-chome, Tsukuba-shi, Ibaraki-ken, Japan) under accession number FERM BP-491 on Feb. 23, 1984) as the template.

In addition, according to the invention, as described in (b) of claims 2, 3 and 5, of the nucleotide sequences set forth in SEQ ID NOs: 1, 3 and 8, a DNA that comprises a nucleotide sequence which hybridizes with a probe comprising a nucleotide sequence consisting of nucleotide numbers 301 to 2073 of SEQ ID NO: 1, a nucleotide sequence consisting of nucleotide numbers 331 to 2154 of SEQ ID NO: 3, or a nucleotide sequence containing at least a part of the foregoing nucleotide sequences under stringent conditions, and that encodes a protein which is capable of enhancing tolerance to acetic acid can be also used as the gene conferring tolerance to short chain fatty acid.

Furthermore, as described in (b), (c) or (d) of claim 4, (b), a DNA that comprises both a nucleotide sequence which hybridizes with a probe comprising a nucleotide sequence consisting of nucleotide numbers 1002 to 1724 in the nucleotide sequence set forth in SEQ ID NO: 5 of the Sequence Listing or comprising at least a part of the foregoing nucleotide sequence under stringent conditions, and a nucleotide sequence consisting of nucleotide numbers 1724 to 2500 in the nucleotide sequence set forth in SEQ ID NO: 5 of the Sequence Listing, and that encodes a protein complex which is capable of enhancing tolerance to acetic acid; (c) a DNA that comprises both a nucleotide sequence consisting of nucleotide numbers 1002 to 1724 in the nucleotide sequence set forth in SEQ ID NO: 5 of the Sequence Listing, and a nucleotide sequence which hybridizes with a probe comprising a nucleotide sequence consisting of nucleotide numbers 1724 to 2500 in the nucleotide sequence set forth in SEQ ID NO: 5 of the Sequence Listing or comprising at least a part of the foregoing nucleotide sequences under stringent conditions, and that encodes a protein complex which is capable of enhancing tolerance to acetic acid, or (d) a DNA that comprises both a nucleotide sequence which hybridizes with a probe comprising a nucleotide sequence consisting of nucleotide numbers 1002 to 1724 in the nucleotide sequence set forth in SEQ ID NO: 5 of the Sequence Listing or comprising at last a part of the foregoing nucleotide sequence under stringent conditions, and a nucleotide sequence which hybridizes with a probe comprising a nucleotide sequence consisting of nucleotide numbers 1724 to 2500 in the nucleotide sequence set forth in SEQ ID NO: 5 of the Sequence Listing or comprising at least a part of the foregoing nucleotide sequence under stringent conditions, and that encodes a protein complex which is capable of enhancing tolerance to acetic acid, can also be used likewise.

The term “stringent conditions” used herein refers to conditions where a so-called specific hybridization occurs, and a non-specific hybridization does not occur. Although it is difficult to precisely quantify these conditions, some examples can be presented, such as conditions under which DNAs having high homology with each other, for example, two DNAs having homology of 70% or greater with each other, can be hybridized, and two DNAs having homology lower than 70% with each other are not hybridized; conditions of conventional washing conditions for hybridization, for example, conditions under which washing is performed at 60° C. with a solution, having a salt concentration corresponding to that of 1×SSC comprising 0.1% SDS and the like.

It can be confirmed that the above-mentioned gene conferring tolerance to short chain fatty acid encodes a protein having a function of transporting a short chain fatty acid from the inside of cells to the outside of cells by, for example, as described below in Examples, determining whether cells which are transformed with the gene and induced to express the gene, namely, transformants, can grow when cultured in a medium comprising a short chain fatty acid, or determining an increase in growth rate in that medium.

Here, “short chain fatty acid” means a straight-chained or branched-chained fatty acid having 1 to 5 carbon atoms, where the fatty acid may or may not have unsaturated bonds. The cells obtained by the method of breeding of the invention are cells showing high tolerance to saturated a straight-chained or branched-chained fatty acids having a 1 to 5 carbon atoms, namely, formic acid, acetic acid, propionic acid, butyric acid, isobutyric acid or valeric acid as described in claim 6.

Cells to which the method of breeding of the invention is applicable are not particularly limited, so long as cells are ones which can be transformed with a gene conferring tolerance to a short chain fatty acid, and express it. Examples of cells include microbial cells, including bacterial cells of Escherichia coli, Bacillus subtilis, Lactobacillus and the like; and yeast cells, the microbial cells belonging to genus Aspergillus and the like.

Among them, it is preferable to use microbial cells as the cells as described in claim 7. This is because culture efficiency in industrial production, particularly in high cell-density culture, and production efficiency in the production of a short chain fatty acid by fermentation are improved by enhancing tolerance to a short chain fatty acid.

Examples of the microbial cells include, in particular, as described in claim 8, the bacterial cells belonging to acetic acid bacteria, genus Escherichia or genus Bacillus. Among the bacterial cells, it is preferable to use those bacterial cells having essentially low ability to oxidize alcohol, such as Escherichia coli, acetic acid bacteria of genus Gluconacetobacter, and the like, because it is possible to significantly increase the amount of product or production efficiency of the desired short chain fatty acid and the cell growth.

Examples of acetic acid bacteria include those strains belonging to genera Acetobacter and Gluconacetobacter.

Specific examples of the bacteria belonging to genus Acetobacter include Acetobacter aceti, and in particular, Acetobacter aceti strain No. 1023 (deposited at International Patent Organism Depositary, National Institute of Advanced Industrial Science and Technology (Central 6, 1-1, Higashi 1-chome, Tsukuba-shi, Ibaraki-ken, Japan) (former designation: Fermentation Research Institute Agency of Industrial Science and Technology, Ministry of International Trade and Industry, former address: 1-3, Higashi 1-chome, Tsukuba-shi, Ibaraki-ken, Japan) under accession number FERM BP-2287 on Jun. 27, 1983 (transferred from the original deposit deposited on Feb. 13, 1989), Acetobacter aceti subspecies xylinum strain IF03288, Acetobacter aceti strain IFO3283, and the like.

Examples of the bacteria belonging to genus Gluconacetobacter include Gluconacetobacter europaeus, Gluconacetobacter diazotrophicus, and Gluconacetobacter entanii, and in particular, Gluconacetobacter europaeus strain DSM6160, Gluconacetobacter diazotrophicus strain ATCC49037, Gluconacetobacter entanii, Acetobacter altoacetigenes strain MH-24 (deposited at International Patent Organism Depositary, National Institute of Advanced Industrial Science and Technology (Central 6, 1-1, Higashi 1-chome, Tsukuba-shi, Ibaraki-ken, Japan) (former designation: Fermentation Research Institute Agency of Industrial Science and Technology, Ministry of International Trade and Industry, former address: 1-3, Higashi 1-chome, Tsukuba-shi, Ibaraki-ken, Japan) under accession number FERM BP-491 on Feb. 23, 1984), which is one species of Gluconacetobacter entanii, can be used.

Preferred examples of coliform bacteria include the bacteria belonging to genus Escherichia.

Examples of the bacteria belonging to genus Escherichia include Escherichia coli, and in particular, Escherichia coli strain K12, E. coli strain JM109, E. coli strain DH5α, E. coli strain C600, E. coli strain BL21, E. coli strain W3110 and the like can be used.

Furthermore, examples of the bacteria belonging to genus Bacillus include Bacillus subtilis and Bacillus subtilis (natto), and in particular, Bacillus subtilis strain Marburg 168 and the like can be used.

Examples of the yeast cell that can be used include Saccharomyces cerevisiae, Shizosaccharomyces pombe and the like.

In particular, when DNA claimed in claim 2 or claim 3 of the invention is used as a gene conferring tolerance to a short chain fatty acid, it is preferable to use, among these cells, Escherichia coli or Gluconacetobacter diazotrophicus as the cells. Escherichia coli has inherently very low ability to oxidize alcohol, and Gluconacetobacter diazotrophicus has low ability of acetic acid fermentation even though it is an acetic acid bacterium. Thus, by conferring tolerance to a short chain fatty acid to these microorganisms, their usefulness can be sufficiently increased.

Transformation and expression of a gene conferring tolerance to a short chain fatty acid in cells according to the method of breeding of the invention can be performed using a recombinant vector. That is, this can be performed by a method of amplifying the intracellular copy number of the gene conferring tolerance to a short chain fatty acid by transforming the cells using a recombinant vector constructed by ligating the aforementioned gene to an appropriate vector; or by a method of amplifying the intracellular copy number of the gene conferring tolerance to a short chain fatty acid by transforming the cells using a recombinant vector in which a structural gene conferring tolerance to a short chain fatty acid and a promoter sequence which efficiently functions in the cells are ligated to an appropriate vector.

The vector that can be used for the construction of a recombinant vector may be a phage vector, which can autonomically propagate in the host, or a plasmid vector, or the like.

Examples of the plasmid vector include Escherichia-derived plasmids (for example, pBR322, pBR325, pUC118, pET116b, and the like), Bacillus-derived plasmids (for example, pUB110, pTP5, and the like), yeast-derived plasmids (for example, Yep13, Ycp50, and the like), and the like, while examples of the phage vector include λ phage (λgt10, λZAP, and the like), and the like.

Animal virus vectors such as those derived from retrovirus, vaccinia virus or the like, insect virus vectors such as those derived from baculovirus or the like, bacteria artificial chromosome (BAC), yeast artificial chromosome (YAC) and the like can be used to transform cells.

Also, multicopy vectors, transposons or the like can be used to introduce a desired DNA into the host, and according to the invention, such multicopy vectors or transposons are also to be included in the vectors of the invention.

Examples of the multicopy vectors include pUF106 (see, for example, Cellulose, p. 153-158 (1989)), pMV24 (see, for example, Appl. Environ. Microbiol., Vol. 55, p. 171-176 (1989)), pGI18 (see, for example, Production Example 1 described below), pTA5001(A), pTA5001(B) (see, for example, JP-A No. 60-9488), and the like. Furthermore, pMVL1, a chromosome-integration type vector (see, for example, Agric. Biol. Chem., vol. 52, p. 3125-3129 (1988)), may also be included.

Examples of the transposon include Mu, IS1452 and the like.

For construction of a recombinant vector by ligating the gene conferring tolerance to a short chain fatty acid to a vector, a method of cleaving purified DNA with an appropriate restriction enzyme, and inserting the resulting fragment into a restriction enzyme site or a multicloning site of an appropriate vector DNA to be ligated to the vector, or the like may be employed.

Here, the recombinant vector thus obtained is required to be expressed so as to produce a protein that is encoded by the gene conferring tolerance to a short chain fatty acid and has a function of transporting a short chain fatty acid from the inside of the cell to the outside of the cell, when transformed into a cell. Therefore, in addition to the gene conferring tolerance to a short chain fatty acid and the promoter sequence, if desired, cis-elements such as an enhancer and the like, a splicing signal, a poly(A) addition signal, a selection marker, a ribosome-binding sequence (SD sequence) and the like may also be inserted by ligating to the recombinant vector.

Here, examples of the selection marker include dihydrofolate reductase gene, kanamycin resistance gene, tetracycline resistance gene, ampicillin resistance gene, neomycin resistance gene, and the like.

In addition, the promoter sequence of the gene conferring tolerance to a short chain fatty acid on the chromosomal DNA may be substituted with another promoter sequence which efficiently functions in acetic acid bacteria belonging to genus Acetobacter or genus Gluconacetobacter, or in Escherichia coli. In this case, a recombinant vector with a DNA sequence homologous to chromosomal DNA may be prepared, and the constructed vector may be used to induce homologous recombination with the chromosome in a cell.

Examples of such promoter sequences include promoter sequences for the ampicillin resistance gene of E. coli plasmid pBR322 (TakaraBio, Inc.), the kanamycin resistance gene of plasmid pHSG298 (TakaraBio, Inc.), the chloramphenicol resistance gene of plasmid pHSG396 (TakaraBio, Inc.), β-galactosidase gene and the like, that are derived from microorganisms other than acetic acid bacteria.

Construction of a vector for homologous recombination is well known to those having ordinary skill in the art. As such, when an endogenous gene that confers tolerance to a short chain fatty acid is placed so as to be expressed under control of a potent promoter in a microorganism, the gene conferring tolerance to a short chain fatty acid is amplified due to multiple copies, and expression thereof is enhanced.

Specific examples of such recombinant vectors include pABC111, pABC112, pABC31 and pABC41, which are respectively obtained by ligating DNAs comprising the nucleotide sequences set forth in SEQ ID NO: 1, 3, 5 and 8 of the Sequence Listing to pGI18, an Acetobacter-Escherichia coli shuttle vector (multicopy vector).

Transformation and expression of the gene conferring tolerance to a short chain fatty acid in cells in the method of breeding of the invention can be performed by the standard methods, using the recombinant vectors described above. For example, in the case of bacterial cells, the method using calcium ions (see, for example, Agric. Biol. Chem., Vol. 49, p. 2091-2097 (1985)), electroporation (see, for example, Proc. Natl. Acad. Sci., U.S.A., Vol. 87, p. 8130-8134 (1990); Biosci. Biotech. Biochem., Vol. 58, p. 974 (1994)), and the like may be used. In the case of using yeast cells as a host, for example, electroporation, a spheroplast method, a lithium acetate method and the like may be used.

Furthermore, the transformant may be selected by the properties conferred by the marker gene contained in the gene to be transformed. For example, in the case of using an ampicillin resistance gene as the marker gene, transformant can be obtained by selecting a cell exhibiting resistance to ampicillin. Specifically, a selection agar plate added with an appropriate amount of ampicillin (for example, about 100 μg/ml) is used, and cells are spread on it and cultured. Colonies that grow on the selection agar plate can be transformants. Also, in the case of using a neomycin resistance gene, a cell exhibiting resistance to the G418 drug can be selected.

According to the method of breeding of the invention, cells that show improved tolerance to a short chain fatty acid can be obtained by transformation of cells with a gene conferring tolerance to a short chain fatty acid and expressing the gene therein, as described above. In the case of bacterial cells, improvement of tolerance to short chain fatty acids of the bred cells can be confirmed as follows: culturing the cells in a medium added with about 0.01 to 3% of a short chain fatty acid for 15 hours or longer, determining the degree of growth by measuring the absorbance or dried cell mass, and comparing the cells with non-transformed original cells, whereby confirming that the transformant shows significantly increased cell mass compared with the original cell that exhibits no growth or insufficient growth. As described later in Examples, the present inventors have obtained cells showing improved tolerance to a short chain fatty acid, such as strain No. 1023/pABC111 obtained by transforming Acetobacter aceti strain No. 1023 with the above-mentioned recombinant vector pABC111; strain JM109/pABC111 obtained by transforming Escherichia coli strain JM109 with the recombinant vector pABC111; strain ATCC49037/pABC111 obtained by transforming Gluconacetobacter diazotrophicus strain ATCC49037 with the recombinant vector pABC111; strain JM109/pABC112 obtained by transforming Escherichia coli strain JM109 with the recombinant vector pABC112; strain JM109/pABC31 obtained by transforming Escherichia coli strain JM109 with the recombinant vector pABC31; and strain JM109/pABC41 obtained by transforming Escherichia coli strain JM109 with the recombinant vector pABC41. Five strains of these transformants are deposited at International Patent Organism Depositary, National Institute of Advanced Industrial Science and Technology (Central 6, 1-1, Higashi 1-chome, Tsukuba-shi, Ibaraki-ken, Japan), and their accession numbers are FERM BP-10184 for the strain JM109/pABC111; FERM BP-10186 for the strain ATCC49037/pABC111; FERM BP-10185 for the strain JM109/pABC112; FERM BP-10182 for the strain JM109/pABC31; and FERM BP-10194 for the strain JM109/pABC41, respectively.

[2] High Cell-Density Culture Method of the Invention

The high cell-density culture method of the invention is characterized in that, as described in claim 9, the cells described in claim 8 are used; in other words, bacterial cells belonging to acetic acid bacteria, genus Escherichia or genus Bacillus showing improved tolerance to short chain fatty acid obtained by introducing the gene conferring tolerance to a short chain fatty acid into bacterial cells and expressing the gene therein, according to the method of breeding of the invention described above in [1].

Here, “high cell-density culture method” means a method of culturing bacterial cells to a high density, that is, to the cell density of 50 to 200 g (dry cell)/L in the medium. The medium, culture period and culture conditions may be appropriately selected according to the type of the cell, or the like. For example, in the case of Escherichia coli, any of complex and synthetic mediums may be used, and for example, the cells can be cultured under aerobic condition in a glucose-added LB medium at 28 to 37° C.

When nutrients in the medium are completely consumed by the bacterial cells, short chain fatty acids are usually produced in culture broth, that causes repression of growth. However, the high cell-density culture method of the invention employs bacterial cells showing improved tolerance to a short chain fatty acid, and thus can prevent repression of growth. Thus, as described in claim 10, even though microbial cells are cultured in the presence of a short chain fatty acid such as formic acid, acetic acid, propionic acid, butyric acid, isobutyric acid, valeric acid or the like which are toxic to cell growth, the growth is not repressed, and the productivity of useful substances produced by the microorganism or recombinant proteins encoded by introduced exogenous gene can be maintained. Here, the useful substance may be any substance that can be produced by microorganisms, and may be in any form and not particularly limited. One example thereof may be the cells of a microorganism itself. Furthermore, when the cells of the invention are used, the final cell density can be increased, and thus any substance, even it is a product as a result of normal metabolic activity, can be produced very efficiently. Examples of such substance include fatty acids, amino acids, antibiotics, enzymes, vitamins, alcohol, and the like. As shown in Example 6, the amount of organic acids produced in the medium can be increased. Also, examples of the recombinant protein include human growth hormones or peptides, interleukin 1β, interferon, and the like.

Particularly, in the case of Escherichia coli, organic acids other than the short chain fatty acids having 5 or fewer carbon atoms, such as citric acid, malic acid, succinic acid, pyroglutamic acid and the like, can be efficiently produced.

Examples of the high cell-density culture method, which is used in various kinds of application from laboratory scale to industrial production scale, include fed-batch culture and dialysis culture.

The fed-batch culture is a method of culture cells while continuously or intermittently feeding specific nutrients to the medium during the culture period. The nutrients to be fed are preferably carbon sources such as glucose, glycerol and the like, which are incorporated and directly used as raw materials for production of short chain fatty acids that inhibit cell growth, or protein production. In order to maintain a constant concentration of the carbon sources in the medium and/or growth rate, feeding may be carried out in a manner where a feeding rate is increased exponentially as the culture proceeds or in a manner where a feeding rate is increased step-wisely (generally at an interval of 2 to 5 hours) with maintaining the concentration of the carbon source in a medium below a predetermined concentration. The culture conditions such as medium composition, culture temperature, humidity, pH, culture time, stirring, and the like may be appropriately selected depending on strain or state of the bacterial cell.

In the conventional methods of fed-batch culture, it was necessary to control the feeding rate of glucose or the like and to control the concentration of dissolved oxygen in the medium minutely for suppression of production of short chain fatty acids, to avoid inhibition of cell growth, repression of protein production or the like, caused by the short chain fatty acids including acetic acid, that are produced during the culture of microorganisms. However, the bacterial cells belonging to acetic acid bacteria, genus Escherichia or genus Bacillus, in which cells show improved tolerance to a short chain fatty acid according to the invention, can maintain productivity without being affected by the short chain fatty acids, and thus the laborious control in the conventional method can be unnecessary.

In addition, the dialysis culture is a method of performing culture while removing extracellular products such as acetic acid to the outside of the culture system by use of dialysis membrane or the like.

While conventional methods of dialysis culture require specialized equipment, the bacterial cells belonging to acetic acid bacteria, genus Escherichia or genus Bacillus, in which cells show improved tolerance to a short chain fatty acid according to the invention, can maintain productivity without being affected by the short chain fatty acids, and thus the culture can be performed using a simple equipment, for example, a jar fermentor.

[3] Preparation of Fermentation Broth Comprising Short Chain Fatty Acids

The method of preparing a fermentation broth according to the invention is characterized in that the cells as described in claim 8 are cultured under the conditions where the cells produce a short chain fatty acid.

The cells as described in claim 8 refer to the bacterial cells belonging to acetic acid bacteria, genus Escherichia or genus Bacillus, in which cells show improved tolerance to a short chain fatty acid, and are obtained by introducing the gene conferring tolerance to a short chain fatty acid into bacterial cells which belong to acetic acid bacteria, genus Escherichia or genus Bacillus, expressing the gene therein and culturing the cells, according to the method of breeding of the invention described above in [1]. Among these cells, those bacterial cells having an ability to produce a desired short chain fatty acid can be selected and used. For example, Escherichia can produce all types of short chain fatty acids, while acetic acid bacteria can selectively produce acetic acid. Furthermore, the short chain fatty acids also include lactic acid, in addition to formic acid, acetic acid, propionic acid, butyric acid, isobutyric acid, and valeric acid.

According to the method of preparing a fermentation broth of the invention, the bacterial cells of the present invention need to be cultured under the conditions where the bacterial cells produce short chain fatty acids, and such conditions may be suitably selected depending on the type of the bacterial cell or the type of the short chain fatty acid to be produced. Generally, a short chain alcohol corresponding to the short chain fatty acid is added to the culture in an appropriate amount.

### EXAMPLES

Hereinafter, the present invention will be described in detail with reference to Examples.

### Production Example 1

### Development of Escherichia coli-Acetobacter shuttle vector pGI18

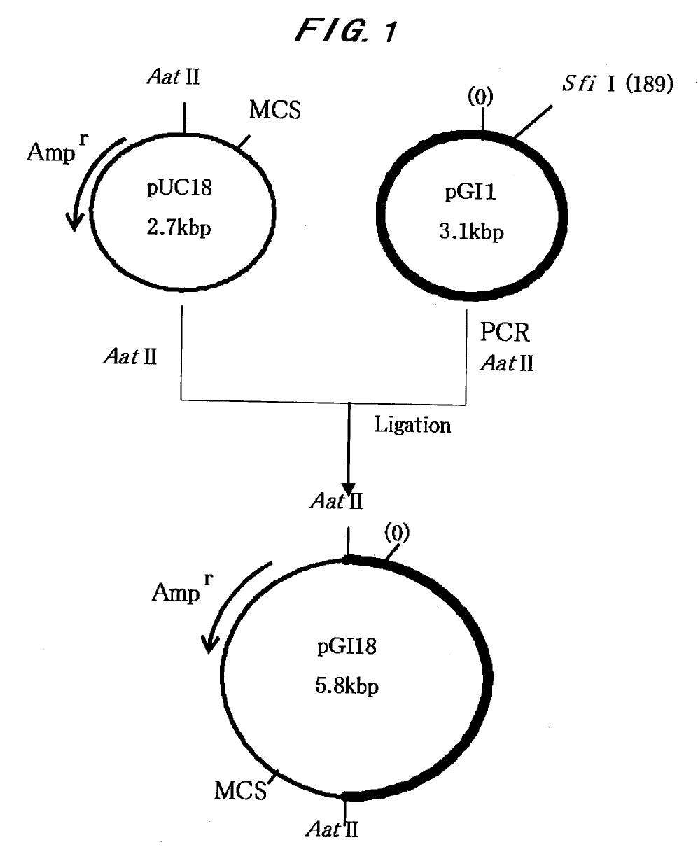

Escherichia coli-Acetobacter shuttle vector pGI18 was constructed from pGI1, a plasmid of about 3.1 kb derived Acetobacter altoacetigenes strain MH-24 (FERM BP-491), and pUC18. FIG. 1 presents a scheme for the construction of the plasmid pGI18.

First, plasmid pGI1 was prepared from Acetobacter altoacetigenes.

That is, the bacteria cells were collected from a culture broth of the Acetobacter altoacetigenes strain MH-24, and the cells were lysed using sodium hydroxide or sodium dodecyl sulfate. Subsequently, the resulting lysate was treated with phenol, and the plasmid DNA was purified using ethanol.

The obtained plasmid was a ring-shaped double-stranded DNA having three recognition sites for HincII and one recognition site for SfiI, and the total length of the plasmid was about 3100 nucleotide pairs (3.1 kbp). There were no recognition sites for EcoRI, SacI, KpnI, SmaI, BamHI, XbaI, SalI, PstI, SphI and HindIII in the plasmid. This plasmid was designated as pGI1 and was used for the construction of the vector pGI18.

The plasmid pGI1 thus obtained was amplified by PCR and the PCR product was cleaved by AatII. The fragment thus obtained was inserted into the cleavage site of pUC18 (2.7 kbp, Takara Bio, Inc.) digested with AatII to construct the plasmid pGI18.

In detail, the PCR was performed as follows. The plasmid pGI1 was used as the template, and primer A (see the nucleotide sequence described in SEQ ID NO: 10 of the Sequence Listing) and primer B (see the nucleotide sequence described in SEQ ID NO: 11 of the Sequence Listing), both comprising recognition site for restriction enzyme AatII, were used as primers. Thirty cycles of the PCR was performed using the template and primers described above and KOD-Plus (Toyobo Co., Ltd.), each cycle consisting of heating at 94° C. for 30 seconds, 60° C. for 30 seconds and 68° C. for 3 minutes.

The resulting plasmid pGI18 contained, as shown in FIG. 1, both pUC18 and pGI1, and the full length was about 5800 nucleotide pairs (5.8 kbp).

The nucleotide sequence of this plasmid pGI18 was shown in SEQ ID NO: 12 of the Sequence Listing.

### Example 1

### Transport of Acetic Acid by Acetobacter aceti Transformed with DNA Comprising Nucleotide Sequence Set Forth in SEQ ID NO: 1 of Sequence Listing

(1) Preparation of Transformant

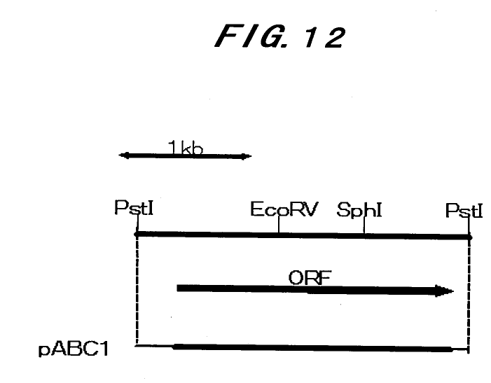

pABC1 (FIG. 12) into which a DNA comprising the nucleotide sequence set forth in SEQ ID NO: 1 of the Sequence Listing was inserted, was cleaved with restriction enzyme PstI, and the fragment of about 2.5 kb thus obtained was ligated to the restriction enzyme PstI cleavage site of the Acetobacter-E. coli shuttle vector pGI18 prepared in the Production Example 1, to construct a plasmid pABC111.

The pABC111 thus constructed was used to transform Acetobacter aceti strain No. 1023 (FERM BP-2287) by electroporation (See Proc. Natl. Acad. Sci. U.S.A., Vol. 87, p. 8130-8134 (1990)). The transformant was selected on YPG medium added with 100 μg/ml of ampicillin and 2% of acetic acid.

The plasmid DNA was extracted from cells of the ampicillin-resistant transformant which were cultured in the above mentioned selection medium and analyzed by the standard method, and it was confirmed that the transformed cells carried a plasmid comprising the DNA comprising the nucleotide sequence set forth in SEQ ID NO: 1 of the Sequence Listing.

(2) Acetic Acid Transport in Transformant

The resulting transformant (strain No. 1023/pABC111) carrying the plasmid pABC111 and showing tolerance to ampicillin was grown in YPG medium added with acetic acid, and was compared with Acetobacter aceti strain No. 1023 (strain No. 1023/pGI18) transformed with the shuttle vector pGI18, in terms of the intracellular acetic acid concentration.

That is, strain No. 1023/pABC111 and strain No. 1023/pGI18 were cultured in 100 ml each of YPG medium added with potassium acetate at concentrations of 1, 2, 3 or 4% (w/v), according to the method of Stainer et al. (see, for example, Biotechnol. Bioeng., Vol. 84, p. 40-44, 2003). The bacterial cells were collected from 50 ml of the culture broth and lysed with alkali, and then the intracellular acetic acid concentration (M) of cells obtained from each culture was measured using F-kit (Roche) and compared.

The results of the intracellular potassium acetate concentration in each culture are presented in Table 1.

TABLE 1

| | Potassium acetate concentration (% (w/v)) | | | |
|---|---|---|---|---|
| | 1 | 2 | 3 | 4 |
| Strain No. 1023/pGI18 | 1.27 M | 3.41 M | 5.31 M | 7.80 M |
| Strain No. 1023/pABC111 | 1.24 M | 3.16 M | 4.25 M | 6.59 M |

As shown in Table 1, the intracellular acetate concentration of the transformant strain No. 1023/pABC111 and the non-transformed strain No. 1023/pGI18 both increased with increasing potassium acetate concentration. However, the degree of increase was relatively low in the transformant (strain No. 1023/pABC111), and in particular, in the presence of 4% (w/v) of potassium acetate, strain No. 1023/pABC111 accumulated acetate inside the cell, the amount of which was only 84% of that of the strain No. 1023/pGI18. This shows that the intracellular acetic acid was transported to the outside of the bacterial cells in the transformant strain No. 1023/pABC111.

From the results, it was confirmed that the DNA comprising the nucleotide sequence set forth in SEQ ID NO: 1 of the Sequence Listing was a gene encoding a protein with a function of transporting acetic acid from the inside of the cells to the outside of the cells.

### Example 2

### Tolerance to Short Chain Fatty Acid of E. coli Transformed with DNA Comprising Nucleotide Sequence Set Forth in SEQ ID NO: 1 of Sequence Listing

(1) Preparation of Transformant

E. coli strain JM109 was transformed with pABC111 constructed in Example 1 by electroporation according to the standard method, and the transformant was selected on the LB agar medium added with 100 μg/ml of ampicillin.

From cells of the ampicillin-resistant transformant, which had been grown in the selective medium, the plasmid DNA was extracted and analyzed by the standard method, and it was confirmed that the transformant carried a plasmid comprising the gene conferring tolerance to acetic acid.

The resulting transformant (strain JM109/pABC111) was deposited at International Patent Organism Depositary, National Institute of Advanced Industrial Science and Technology (Central 6, 1-1, Higashi 1-chome, Tsukuba-shi, Ibaraki-ken, Japan) on Dec. 14, 2004 under Deposit No. FERM BP-10184.

(2) Tolerance to Short Chain Fatty Acid of Transformant

The resulting ampicillin-resistant transformant (strain JM109/pABC111), which carried the plasmid pABC111, was compared with E. coli (strain JM109/pGI18) carrying the shuttle vector pGI18 in terms of the growth in the LB medium which was added with formic acid, acetic acid, propionic acid, butyric acid, isobutyric acid, or valeric acid, respectively as a short chain fatty acid.

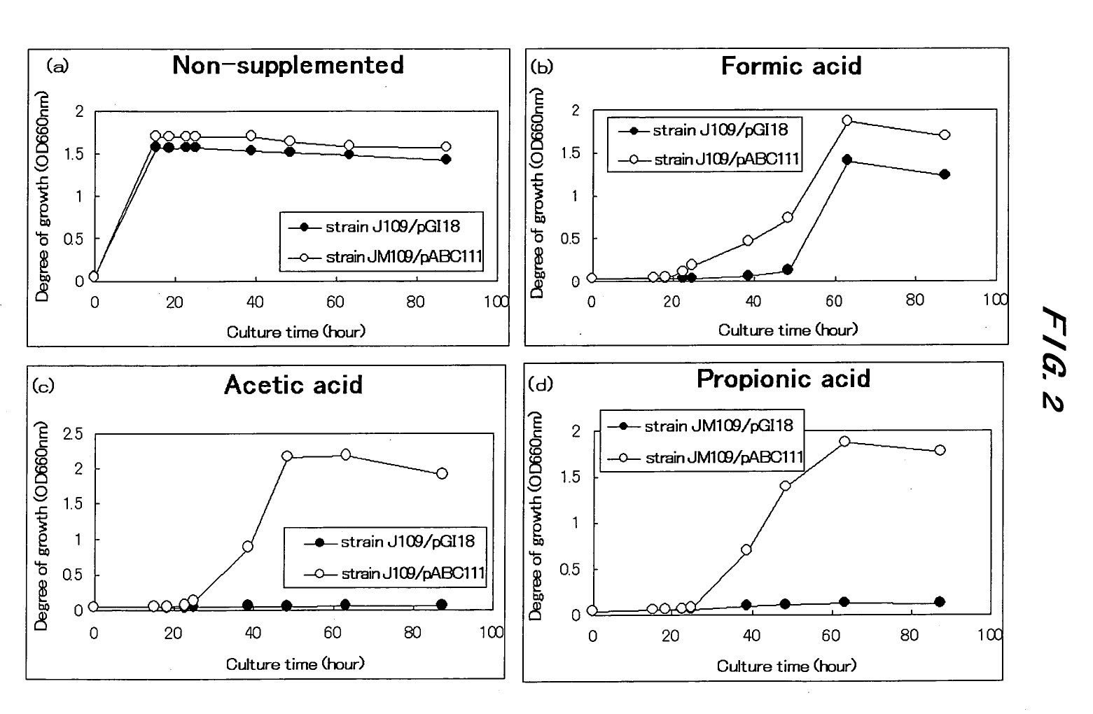

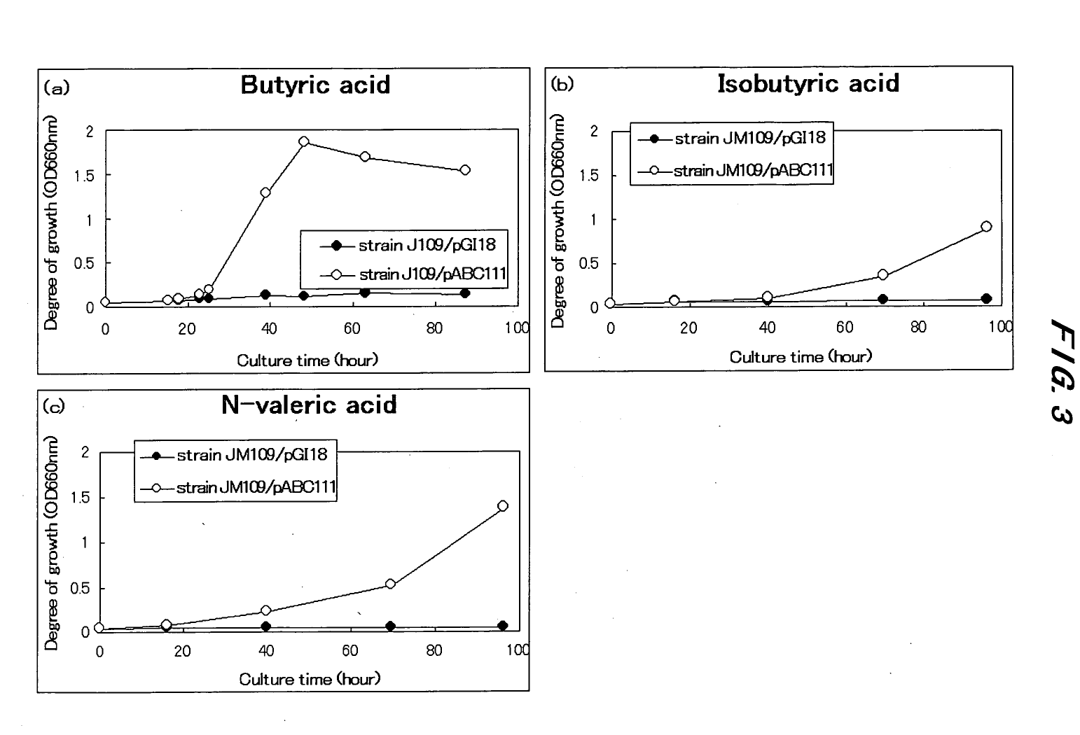

That is, strain JM109/pABC111 and strain JM109/pGI18 were cultured with shaking (150 rpm) in a LB medium (pH 5.0: a medium added with a short chain fatty acid) comprising any one of formic acid 0.10%, acetic acid 0.15%, propionic acid 0.10%, butyric acid 0.10%, isobutyric acid 0.15%, and n-valeric acid 0.25% as a short chain fatty acid, 100 μg/ml of ampicillin, and 1 mM IPTG (isopropyl-1-thio-β-D-galactopyranoside) at 37° C. Further, for comparison, they were cultured similarly as described above, except that a short chain fatty acid was not added to the medium. In each medium, absorbance of the broth, indicative of the growth (cell mass), was measured at 660 nm during culture and the measured values of absorbance were compared. FIGS. 2 and 3 show the time course in growth (absorbance at 660 nm) during culture in each medium.

As shown in FIGS. 2 and 3, both cells grew in the non-supplemented medium, on the other hand, strain JM109/pGI18 which did not carry the subject gene did not grow, or not substantially grow in the medium added with a short chain fatty acid, but the strain JM109/pABC111 which was transformed with the subject gene grew in the medium added with a short chain fatty acid. That is, the growth of non-transformed strain (strain JM109/pGI18) was affected by the presence of the short chain fatty acid, but the transformant (strain JM109/pABC111) was tolerant to all the short chain fatty acids tested.

This demonstrates that E. coli showing improved tolerance to various short chain fatty acids can be obtained by introducing DNA comprising the nucleotide sequences set forth in SEQ ID NO: 1 of Sequence Listing into the cells.

### Example 3

### Tolerance to Short Chain Fatty Acid of E. coli Transformed with DNA Comprising Nucleotide Sequence Set Forth in SEQ ID NO: 3 of Sequence Listing

(1) Preparation of Transformant

The DNA comprising the nucleotide sequences set forth in SEQ ID NO: 3 of Sequence Listing was amplified by a PCR process, and the obtained fragment was inserted into the site cleaved by restriction enzyme SmaI cleavage site of the Acetobacter-E. coli shuttle vector pGI18 prepared as in Production Example 1, to construct a plasmid pABC112.

Specifically, the PCR process was performed as follows. The genomic DNA of Acetobacter altoacetigenes strain MH-24 (FERM BP-491) was used as the template, and primer 1 (see the nucleotide sequence set forth in SEQ ID NO: 13 of Sequence Listing) and primer 2 (see the nucleotide sequence set forth in SEQ ID NO: 14 of Sequence Listing) were used as primers. Thirty cycles of PCR was performed using the template and primers described above and KOD-Plus (Toyobo Co., Ltd.), each cycle consisting of heating at 94° C. for 15 seconds, 60° C. for 30 seconds, and 68° C. for 2 minutes.

E. coli (Escherichia coli) stain JM109 was transformed with the resulting pABC112 by electroporation (see Biosci. Biotech. Biochem., Vol. 58, p. 974 (1994)). The transformant was selected on the LB agar medium added with 100 μg/ml of ampicillin.

The plasmid DNA was extracted from cells of the ampicillin-resistant transformant which were cultured in the above mentioned selection medium and analyzed by the standard method, and it was confirmed that the transformant carried a plasmid comprising the DNA comprising the nucleotide sequence set forth in SEQ ID NO: 3 of Sequence Listing.

The resulting transformant (strain JM109/pABC112) was deposited at International Patent Organism Depositary, National Institute of Advanced Industrial Science and Technology (Central 6, 1-1, Higashi 1-chome, Tsukuba-shi, Ibaraki-ken, Japan) on Dec. 14, 2004 under Deposit No. FERM BP-10185.

(2) Tolerance to Short Chain Fatty Acid of Transformant

The resulting ampicillin-resistant transformant (strain JM109/pABC112), which carried the plasmid pABC112, was compared with E. coli (strain JM109/pGI18) carrying the shuttle vector pGI18 in terms of the growth in the LB medium added with formic acid or acetic acid as a short chain fatty acid.

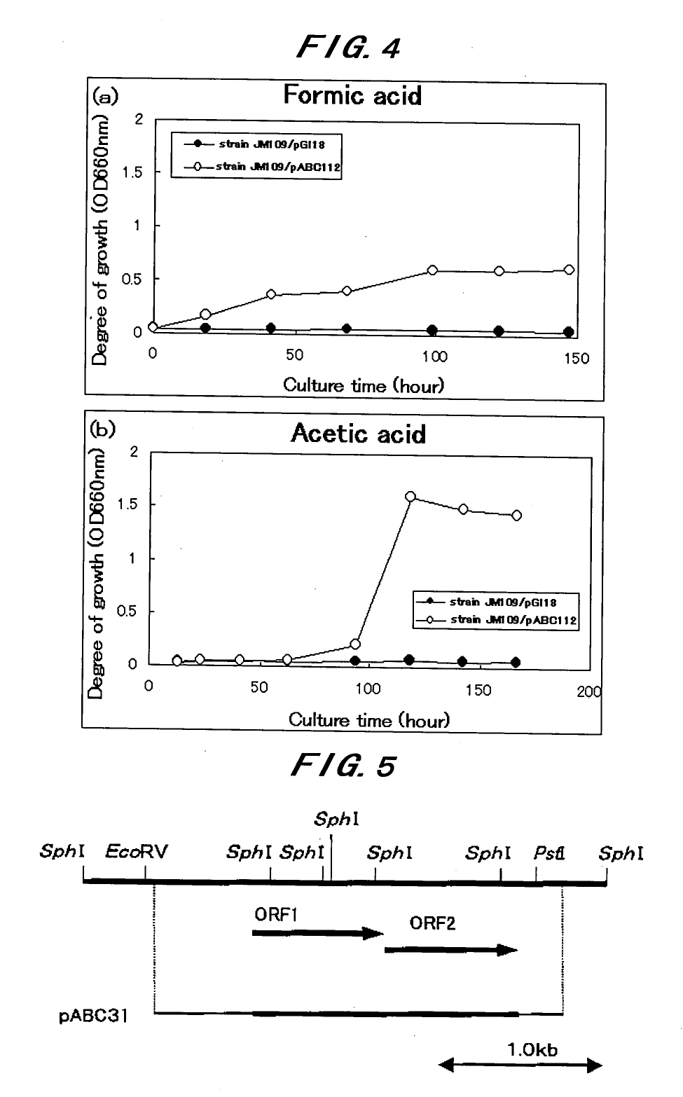

Specifically, strain JM109/pABC112 and strain JM109/pGI18 were cultured without or with shaking (150 rpm) in a LB medium (pH 5.0) comprising 0.10% of formic acid, or 0.15% of acetic acid as a short chain fatty acid, 100 μg/ml of ampicillin, and 1 mM IPTG (isopropyl-1-thio-β-D-galactopyranoside) at 37° C. In each medium added with a short chain fatty acid, absorbance of the broth, indicative of the growth (cell mass), was measured at 660 nm during culture and the measured values of absorbance were compared. FIG. 4 shows the time course in growth (absorbance at 660 nm) during culture in each medium.

As shown in FIG. 4, strain JM109/pABC112 which was the transformant grew in any of the media added with a short chain fatty acid, but strain JM109/pABC18 which did not carried the subject gene did not grew in both media added with a short chain fatty acid. That is, the growth of non-transformed strain (strain JM109/pGI18) was affected by the presence of the short chain fatty acid, but the transformant (strain JM109/pABC112) showed tolerance to both formic acid and acetic acid.

This demonstrates that E. coli showing improved tolerance to a short chain fatty acids can be obtained by introducing DNA comprising the nucleotide sequences set forth in SEQ ID NO: 3 of Sequence Listing into the cells of E. coli.

### Example 4

### Tolerance to Short Chain Fatty Acid of E. coli Transformed with DNA Comprising Nucleotide Sequence Set Forth in SEQ ID NO: 5 of Sequence Listing

(1) Preparation of E. coli Transformant

The DNA comprising the nucleotide sequences set forth in SEQ ID NO: 5 of Sequence Listing was amplified by a PCR process, and the obtained fragment was ligated into the site cleaved by restriction enzyme SmaI cleavage site of the Acetobacter-E. coli shuttle vector pGI18 prepared in Production Example 1, to construct a plasmid pABC31.

Specifically, the PCR process was performed as follows. The genomic DNA of Acetobacter altoacetigenes strain MH-24 (FERM BP-491) was used as the template, and primer 3 (see the nucleotide sequence set forth in SEQ ID NO: 15 of Sequence Listing), and primer 4 (see the nucleotide sequence set forth in SEQ ID NO: 16 of Sequence Listing) were used as the primers. Thirty cycles of PCR was performed using the template and primers described above and KOD-Plus (Toyobo Co., Ltd.), each cycle consisting of heating at 94° C. for 15 seconds, 60° C. for 30 seconds, and 68° C. for 1 minute.

For the genomic DNA of Acetobacter altoacetigenes strain MH-24, the restriction enzyme map of the nucleotide sequence set forth in SEQ ID NO: 5, the position of the nucleotide sequence set forth in SEQ ID NO: 5, and the schematic diagram of the fragment inserted into the plasmid pABC31 are presented in FIG. 5.

E. coli (Escherichia coli) strain JM109 was transformed with the resulting pABC31 by electroporation. The transformant was selected on the LB agar medium added with 100 μg/ml of ampicillin.

The plasmid DNA was extracted from cells of the ampicillin-resistant transformant which were cultured in the above mentioned selection medium and analyzed by the standard method. It was confirmed that the transformant carried a plasmid comprising DNA comprising the nucleotide sequence set forth in SEQ ID NO: 5 of Sequence Listing.

The resulting transformant (strain JM109/pABC31) was deposited at International Patent Organism Depositary, National Institute of Advanced Industrial Science and Technology (Central 6, 1-1, Higashi 1-chome, Tsukuba-shi, Ibaraki-ken, Japan) on Dec. 14, 2004 under Deposit No. FERM BP-10182.

(2) Tolerance to Short Chain Fatty Acid of Transformant

The resulting ampicillin-resistant transformant (strain JM109/pABC31), which carried the plasmid pABC31, was compared with E. coli (strain JM109/pGI18) carrying the shuttle vector pGI18 in terms of the growth in the LB medium added with formic acid or acetic acid as a short chain fatty acid.

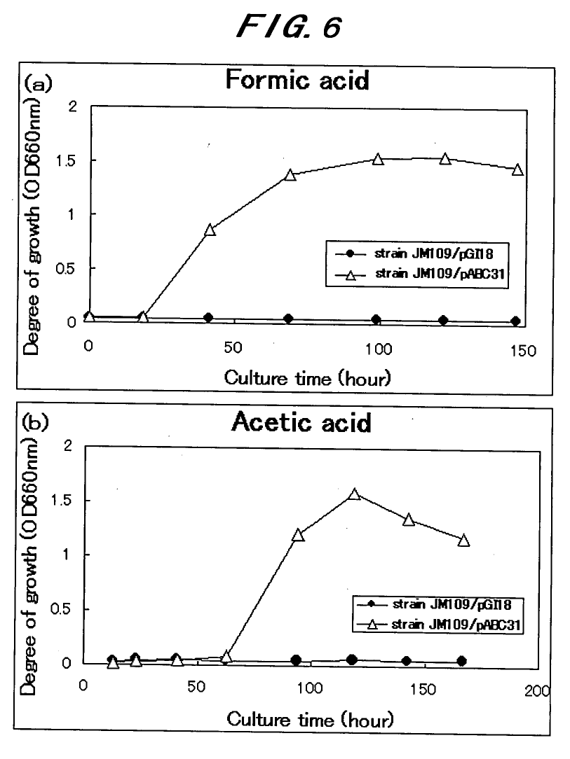

Specifically, strain JM109/pABC31 and strain JM109/pGI18 were cultured without or with shaking (150 rpm) in a LB medium (pH 5.0) added with 0.10% of formic acid or 0.15% of acetic acid as a short chain fatty acid, 100 μg/ml of ampicillin, and 1 mM IPTG (isopropyl-1-thio-β-D-galactopyranoside) at 37° C. In each medium added with a short chain fatty acid, absorbance of the broth, indicative of the growth (cell mass), was measured at 660 nm during culture and the measured values of absorbance were compared. FIG. 6 shows the time course in growth (absorbance at 660 nm) during culture in each medium.

As shown in FIG. 6, strain JM109/pABC31 that was the transformant grew in any of the media added with a short chain fatty acid, but the E. coli strain JM109/pGI18 did not grow in the mediums added with short chain fatty acid. That is, the growth of non-transformed strain (strain JM109/pGI18) was affected by the presence of the short chain fatty acid, but the transformant (strain JM109/pABC31) showed tolerance to both formic acid and acetic acid.

This demonstrates that E. coli showing improved tolerance to a short chain fatty acid can be obtained by introducing DNA comprising the nucleotide sequences set forth in SEQ ID NO: 5 of Sequence Listing into the cells of E. coli.

### Example 5

### Tolerance to Short Chain Fatty Acid of E. coli Transformed with DNA Comprising Nucleotide Sequence Set Forth in SEQ ID NO: 8 of Sequence Listing

(1) Preparation of E. coli Transformant

The DNA comprising the nucleotide sequences set forth in SEQ ID NO: 8 of Sequence Listing was amplified by a PCR process, and the obtained fragment was ligated into the site cleaved by restriction enzyme SmaI cleavage site of the Acetobacter-E. coli shuttle vector pGI18 prepared in Production Example 1, to construct a plasmid pABC41.

Specifically, the PCR process was performed as follows. The genomic DNA of Acetobacter altoacetigenes strain MH-24 (FERM BP-491) was used as the template, and primer 5 (see the nucleotide sequence set forth in SEQ ID NO: 17 of Sequence Listing) and primer 6 (see the nucleotide sequence set forth in SEQ ID NO: 18 of Sequence Listing) were used as the primers. Thirty cycles of PCR were performed using the template and primers described above and KOD-Plus (Toyobo Co., Ltd.), each cycle consisting of heating at 94° C. for 15 seconds, 60° C. for 30 seconds, and 68° C. for 1 minute.

E. coli (Escherichia coli) strain JM109 was transformed with the resulting pABC41 by electroporation. The transformant was selected on the LB agar medium added with 100 μg/ml of ampicillin.

The plasmid DNA was extracted from cells of the ampicillin-resistant transformant which were cultured in the above mentioned selection medium and analyzed by the standard method, and it was confirmed that the transformant carried a plasmid comprising the DNA comprising the nucleotide sequence set forth in SEQ ID NO: 8 of Sequence Listing.

It was confirmed that there was an open-reading-frame (ORF) encoding the amino acid sequence consisting of 259 amino acids set forth in SEQ ID NO: 9, from the nucleotide numbers 249 through 1025, among the nucleotide sequences set forth in SEQ ID NO: 8 of Sequence Listing. Further, the homology of the amino acid sequence set forth in SEQ ID NO: 9 of Sequence Listing with known sequences was as low as 30% at most, indicating that the gene comprising the nucleotide sequences set forth in SEQ ID NO: 8 of Sequence Listing was a novel gene, first discovered by the present inventors.

For the genomic DNA of Acetobacter altoacetigenes MH-24 strain, the restriction enzyme map of the nucleotide sequence set forth in SEQ ID NO: 8, the position of the nucleotide sequence set forth in SEQ ID NO: 8, and the schematic diagram of the fragment inserted into the plasmid pABC41 are presented in FIG. 8.

The resulting transformant (strain JM109/pABC41) was deposited at International Patent Organism Depositary, National Institute of Advanced Industrial Science and Technology (Central 6, 1-1, Higashi 1-chome, Tsukuba-shi, Ibaraki-ken, Japan) on Dec. 27, 2004 under Deposit No. FERM BP-10194.

(2) Tolerance to Short Chain Fatty Acid of Transformant

The resulting ampicillin-resistant transformant (strain JM109/pABC41), which carried the plasmid pABC41, was compared with E. coli (strain JM109/pGI18) carrying the shuttle vector pGI18, in terms of the growth in the LB medium added with formic acid or acetic acid as a short chain fatty acid.

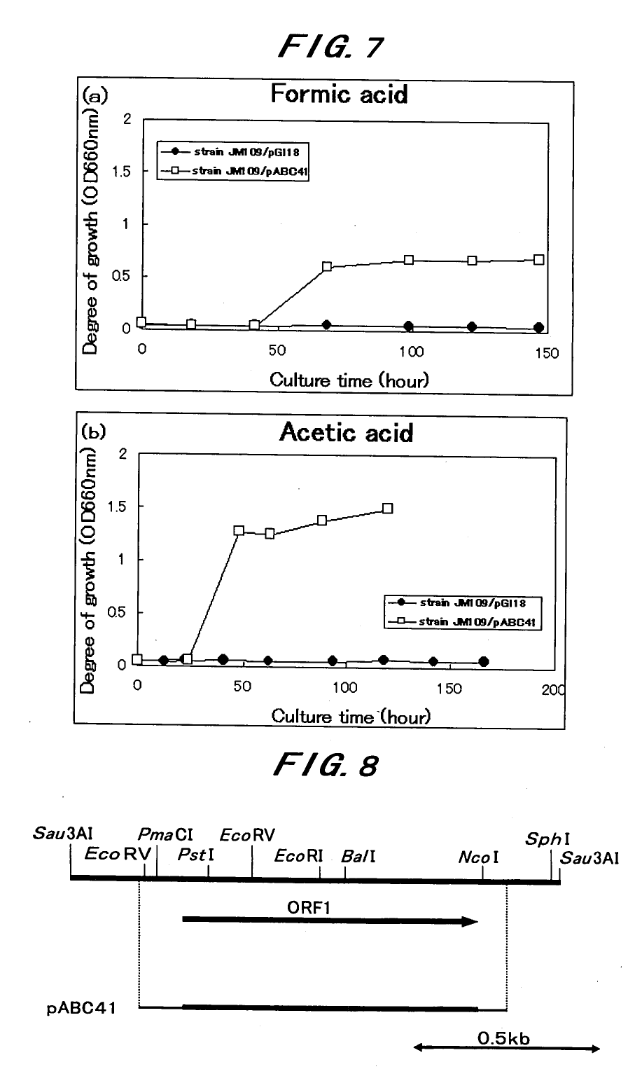

Specifically, the strain JM109/pABC41 and the strain JM109/pGI18 were cultured without or with shaking (150 rpm) in a LB medium (pH 5.0) comprising 0.15% of formic acid or acetic acid as a short chain fatty acid, 100 μg/ml of ampicillin, and 1 mM IPTG (isopropyl-1-thio-β-D-galactopyranoside) at 37° C. In each medium added with a short chain fatty acid, absorbance of the broth, indicative of the growth (cell mass), was measured at 660 nm during culture and the measured values of absorbance were compared. FIG. 7 shows the time course in growth (absorbance at 660 nm) during culture in each medium.

As shown in FIG. 7, the strain JM109/pABC41 which was a transformant grew in any of the media added with a short chain fatty acid, but the E. coli strain JM109/pGI18 did not grow in the medium added with a short chain fatty acid. That is, the growth of the non-transformed strain (strain JM109/pGI18) was affected by the presence of the short chain fatty acid, but the transformant (strain JM109/pABC41) showed tolerance to both formic acid and acetic acid.

This demonstrates that E. coli showing improved tolerance to a short chain fatty acid can be obtained by introducing DNA comprising the nucleotide sequences set forth in SEQ ID NO: 8 of Sequence Listing into the cells of E. coli.

### Example 6

### High Cell-Density Culture of E. coli Transformant

(1) Culture in Glucose-Supplemented LB Medium

The transformant (strain JM109/pABC111) of E. coli strain JM109 comprising the plasmid pABC111 obtained in Example 2 was compared with E. coli (strain JM109/pGI18) carrying the shuttle vector pGI18, in terms of growth in the LB medium added with glucose.

Specifically, the strains were cultured aerobically (0.3 vvm) in a membrane filter-sterilized LB medium (pH 7.0) comprising 20% of glucose, and 100 μg/ml of ampicillin, using a 5-liter mini-jar fermentor (KMJ-2A manufactured by Mitsuwa Scientific Corp.), at 37° C. and 500 rpm. The growth, the amount of acetic acid in broth, and the total amount of organic acids of the transformant and the non-transformant during culture or after the completion of culture were compared.

The growth was determined by measuring the absorbance (OD 660 nm) at 660 nm. The total amount of organic acids and the amount of the acetic acid in the culture were measured by Shimadzu High-Speed Liquid Chromatography Organic Acid Analysis System (Column: ShodexRSpak KC-811, Mobile Phase: 4 mM p-toluenesulfonic acid, Reaction solution: 4 mM p-toluenesulfonic acid, 80 μM EDTA, 16 mM Bis-Tris), using an appropriately diluted samples of supernatant prepared by centrifugation of the broth, and the total amount of organic acids and amount of acetic acid were calculated based on the sum of concentrations of citric acid, malic acid, succinic acid, lactic acid, acetic acid, and pyroglutamic acid, and the concentration of acetic acid, respectively.

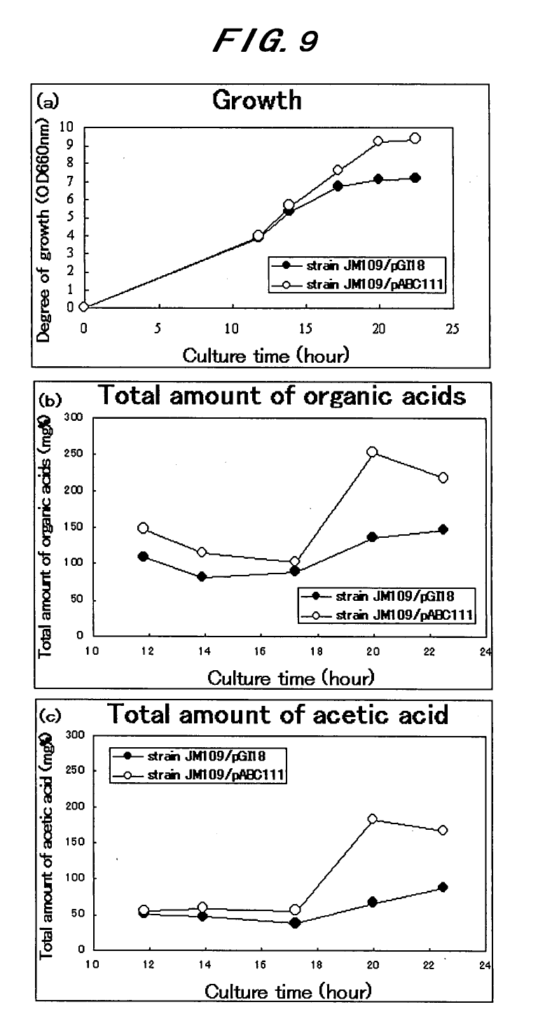

Time courses of the growth amount of each cell, the total amount of organic acids, and the amount of the acetic acid in the culture are presented in FIG. 9. Further, the growth amount at 22 hr after start of the culture, and the concentrations of the total amount of short chain fatty acids and the amount of acetic acid in the culture broth are summarized in Table 2.

TABLE 2

| | Growth | Total concentration of | Concentration |
|---|---|---|---|
| | amount | the short chain fatty | of acetic acid |
| Strain | (OD 660) | acids (mg %) | (mg %) |
| strain JM109/pGI18 | 7.18 | 146.5 | 87.9 |
| strain | 9.34 | 217.6 | 167.0 |
| JM109/pABC111 | | | |

From the results of FIG. 9, it was confirmed that the growth amounts, the total amount of short chain fatty acids (organic acids), and the amount of acetic acid of the transformant (strain JM109/pABC111) were higher than those of the non-transformant (strain JM109/pGI18 strain).

Further, it was confirmed from the results of Table 2 that the growth amount at 22 hr after start of the culture, the total amount of the short chain fatty acids and the amount of acetic acid of transformant (strain JM109/pABC111) were higher than those of the non-transformant (strain JM109/pGI18).

From these findings, it was evident that E. coli obtained by introducing DNA comprising the nucleotide sequence set forth in SEQ ID NO: 1 of Sequence Listing was not adversely affected by the short chain fatty acid in the high cell-density culture, and that organic acids, as well as short chain fatty acids including acetic acid, could be produced in a large amount.

(2) Glucose Fed-Batch Culture

The transformant (strain JM109/pABC111) of E. coli strain JM109 comprising the plasmid pABC111 obtained in Example 2 was compared with E. coli (strain JM109/pGI18) carrying the shuttle vector pGI18, in terms of the growth in the fed-batch culture where glucose was fed during culture.

For the culture, a 2-liter mini-jar fermentor (KMJ-2A manufactured by Mitsuwa Scientific Corp.) and a pH controller (Digital pH controller MPH-2C manufactured by Mitsuwa Scientific Corp.) were used.

Further, as a culture medium, a medium prepared by adding 3 ml of 1 M MgSO₄ and 3 ml of trace element solution (0.5 g of CaCl₂, 0.18 g of ZnSO₄.7H₂O, 0.1 g of MnSO₄.H₂O, 20.1 g of EDTA-Na₂, 16.7 g of FeCl₃.6H₂O, 0.16 g of CuSO₄.5H₂O, 0.18 g of CoCl₂.6H₂O (per liter of culture medium)) to 1 liter of Mineral medium (2.0 g of Na₂SO₄, 2.468 g of (NH₄)₂SO₄, 0.5 g of NH₄Cl, 14.6 g of K₂HPO₄, 3.6 g of NaH₂PO₄.H₂O, 1.0 g of (NH₄)₂-H-citrate, 0.05 g of Thiamin/l)) was used.

Culture was performed while controlling temperature, pH, number of stirring, and aeration at 35° C., 6.8, 700 rpm, and 0.5 l/min, respectively. The pH was adjusted by adding 29% solution of ammonia.

Cells were collected from the culture broth which had been cultured for 6 hours in 5 ml of the LB medium comprising 100 μg/ml of ampicillin, and then suspended in 5 ml of the medium. The resultant suspension was inoculated into 1 liter of the medium, and then aerobically cultured under stirring for 16.5 hours.

Thereafter, cells were aerobically cultured with stirring (aeration amount: 0.5 vvm, and stirring rate: 700 rpm) with feeding 50% solution of glucose until 26.5 hr, the feeding rate and feeding amount being increased as the fermentation proceeds, as shown in Table 3.

TABLE 3

| | Feeding | Feeding |
|---|---|---|
| Culture time (h) | rate (ml/h) | amount (ml) |
| 0 to 16.5 | 0 | 0 |
| 16.5 to 18.5 | 10 | 20 |
| 18.5 to 20.5 | 20 | 40 |
| 20.5 to 22.5 | 30 | 60 |
| 22.5 to 24.5 | 40 | 80 |
| 24.5 to 26.5 | 50 | 100 |

The growth was confirmed by measuring the absorbance at 660 nm (OD 660 nm) and dried cell mass.

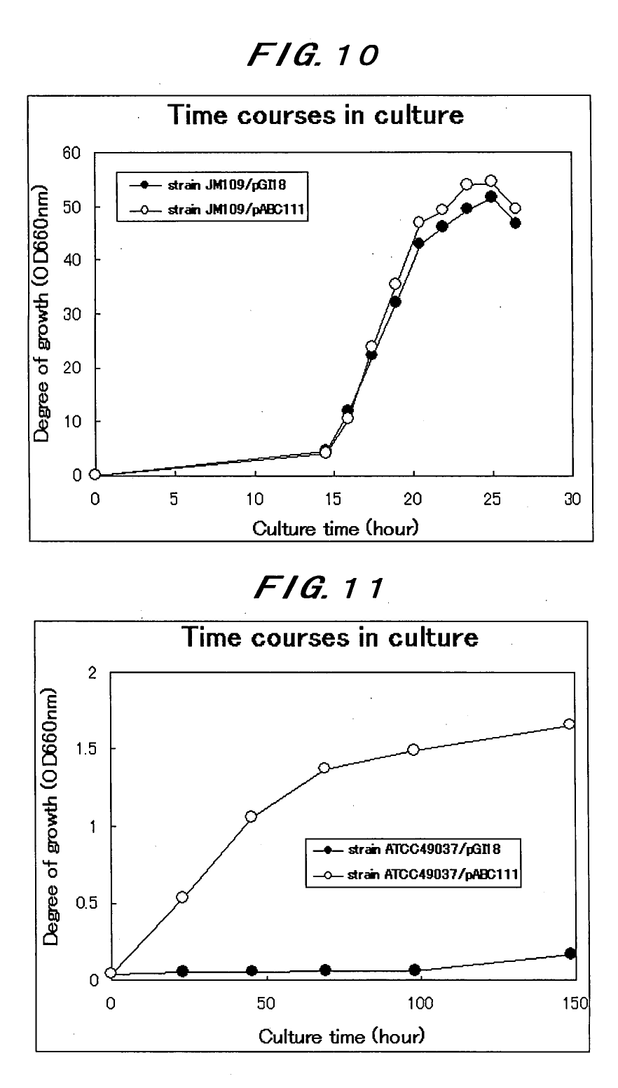

Time course in growth (OD 660 nm) in the glucose fed-batch culture is shown in FIG. 10.

As FIG. 10 evidently shows, it was confirmed that the growth of the transformant (strain JM109/pABC111) was higher than that of the strain JM109/pGI18.

Further, at the end of the culture, the final cell mass (dried cell weight (g: DCW) and the amount of consumed glucose were measured, and based on the measured values, the cell concentration (g/l, dried cell weight per 1 L of medium), and the yield of cell mass (w/w %) were calculated. The growth rate per unit time as the average growth rate (g/h) were determined. The results for each strain were summarized in Table 4.

TABLE 4

| | strain | strain |
|---|---|---|
| | JM109/pGI18 | JM109/pABC111 |
| Final cell mass (g) | 25.74 | 27.05 |
| Cell concentration | 19.92 | 20.38 |
| (g/l) | | |
| Cell mass yield | 10.77 | 11.04 |
| (w/w %) | | |
| Average growth rate | 0.095 | 0.100 |
| (g/h) | | |

From the results of Table 4, it was confirmed that the cell mass, cell concentration, yield of cell mass and growth rate per unit time of transformant (strain JM109/pABC111) were higher than those of the strain JM109/pGI18.

Accordingly, the usefulness of the high cell-density culture using the glucose fed-batch culture was confirmed.

### Example 7

### Tolerance to Short Chain Fatty Acid of Gluconacetobacter diazotrophicus Transformed with DNA Comprising Nucleotide Sequence set forth in SEQ ID NO: 1 of Sequence Listing

(1) Preparation of Transformant

Gluconacetobacter diazotrophicus strain ATCC49037 as one of acetic acid bacteria having no ability of acetic acid fermentation was transformed with the pABC111 constructed in Example 1 by electroporation. The transformant was selected on the YPG agar medium added with 100 μg/ml of ampicillin.

The plasmid DNA was extracted from cells of the ampicillin-resistant transformant which were cultured in the above mentioned selection medium and analyzed by the standard method, and it was confirmed that the transformant carried a plasmid comprising DNA comprising nucleotide sequence set forth in SEQ ID NO: 1 of Sequence Listing

The resulting transformant (strain ATCC49037/pABC111) was deposited at International Patent Organism Depositary, National Institute of Advanced Industrial Science and Technology (Central 6, 1-1, Higashi 1-chome, Tsukuba-shi, Ibaraki-ken, Japan) on Dec. 14, 2004 under Deposit No. FERM BP-10186.

(2) Tolerance to Acetic Acid of Transformant

The resulting ampicillin-resistant transformant (strain ATCC49037/pABC111 strain), which carried the plasmid pABC 111, was compared with the Gluconacetobacter diazotrophicus ATCC49037 strain (JM109/pGI18 strain) carrying the shuttle vector pGI18, in terms of the growth in the LB medium added with acetic acid.

Specifically, the strain was cultured with shaking (150 rpm) in the YPG medium added with 0.05% of acetic acid, and 100 μg/ml of ampicillin at 30° C. The absorbance of the broth, indicative of the growth (cell mass), was measured at 660 nm during culture and the measured values of absorbance were compared. FIG. 11 shows the time course in growth (absorbance at 660 nm) during culture in each medium.

As shown in FIG. 11, it was confirmed that the transformant (strain ATCC49037/pABC111) grew in the YPG medium added with 0.05% of acetic acid, but the strain ATCC49037/pGI18 did not grow in the medium added 0.05% of acetic acid.

This demonstrates that the tolerance to acetic acid of Gluconacetobacter diazotrophicus can be improved by introducing DNA comprising the nucleotide sequences set forth in SEQ ID NO: 1 of Sequence Listing into Gluconacetobacter diazotrophicus having no ability to produce acetic acid.

### INDUSTRIAL APPLICABILITY

According to the present invention, cells to show improved tolerance to a short chain fatty acid can be bred by conferring tolerance to a short chain fatty acid. Furthermore, when the method of breeding of the invention is applied to microbial cells, cells, whose growth is not affected by a short chain fatty acid which is produced during culture and are harmful to growth of the cell, can be bred efficiently.

The microbial cells showing tolerance to a short chain fatty acid obtained by the invention can be also applied to high cell-density culture in which short chain fatty acids are produced. Also, the cells can be used in the preparation of fermentation broths comprising short chain fatty acids at a high concentration. Particularly in the case of Escherichia coli to which tolerance to a short chain fatty acid is conferred, or the like, the bacterial cells show significantly improved ability to grow in medium and can efficiently accumulate the short chain fatty acid at a high concentration, thus being industrially useful.

1. A method of breeding cells to improve tolerance to a short chain fatty acid, wherein a gene encoding a protein having a function of transporting a short chain fatty acid from the inside of cells to the outside of cells, is introduced and expressed in the cells.

2. The method of breeding cells according to claim 1, wherein the gene encoding the protein having a function of transporting a short chain fatty acid from the inside of cells to the outside of cells is a DNA represented by the following (a) or (b):
(a) a DNA that comprises a nucleotide sequence consisting of nucleotide numbers 301 to 2073 in the nucleotide sequence set forth in SEQ ID NO: 1 of the Sequence Listing; or
(b) a DNA that comprises a nucleotide sequence which hybridizes with a probe comprising a nucleotide sequence consisting of nucleotide numbers 301 to 2073 in the nucleotide sequence set forth in SEQ ID NO: 1 of the Sequence Listing or comprising at least a part of said nucleotide sequence under stringent conditions, and that encodes a protein which is capable of enhancing tolerance to acetic acid.

3. The method of breeding cells according to claim 1, wherein the gene encoding the protein having a function of transporting a short chain fatty acid from the inside of cells to the outside of cells is a DNA represented by the following (a) or (b):
(a) a DNA that comprises a nucleotide sequence consisting of nucleotide numbers 331 to 2154 in the nucleotide sequence set forth in SEQ ID NO: 3 of the Sequence Listing; or
(b) a DNA that comprises a nucleotide sequence which hybridizes with a probe comprising a nucleotide sequence consisting of nucleotide numbers 331 to 2154 in the nucleotide sequence set forth in SEQ ID NO: 3 of the Sequence Listing or comprising at least a part of said nucleotide sequence under stringent conditions, and that encodes a protein which is capable of enhancing tolerance to acetic acid.

4. The method of breeding cells according to claim 1, wherein the gene encoding the protein having a function of transporting a short chain fatty acid from the inside of cells to the outside of cells is a DNA represented by the following (a), (b), (c) or (d):
(a) a DNA that comprises a nucleotide sequence consisting of nucleotide numbers 1724 to 2500 and a nucleotide sequence consisting of nucleotide numbers 1724 to 2500 in the nucleotide sequence set forth in SEQ ID NO:5 of in the Sequence Listing;
(b) a DNA that comprises both a nucleotide sequence which hybridizes with a probe comprising a nucleotide sequence consisting of nucleotide numbers 1002 to 1724 in the nucleotide sequence set forth in SEQ ID NO:5 of the Sequence Listing or comprising at least a part thereof under stringent conditions, and a nucleotide sequence consisting of nucleotide numbers 1724 to 2500 in the nucleotide sequence set forth in SEQ ID NO:5 of the Sequence Listing, and that encodes a protein complex which is capable of enhancing tolerance to acetic acid;
(c) a DNA that comprises both a nucleotide sequence consisting of nucleotide numbers 1002 to 1724 in the nucleotide sequence set forth in SEQ ID NO:5 of the Sequence Listing, and a nucleotide sequence which hybridizes with a probe comprising a nucleotide sequence consisting of nucleotide numbers 1724 to 2500 in the nucleotide sequence set forth in SEQ ID NO:5 of the Sequence Listing or comprising at least a part thereof under stringent conditions, and that encodes a protein complex which is capable of enhancing tolerance to acetic acid; or
(d) a DNA that comprises both a nucleotide sequence which hybridizes with a probe comprising a nucleotide sequence consisting of nucleotide numbers 1002 to 1724 in the nucleotide sequence set forth in SEQ ID NO:5 of the Sequence Listing or comprising at least a part thereof under stringent conditions and a nucleotide sequence which hybridizes with a probe comprising a nucleotide sequence consisting of nucleotide numbers 1724 to 2500 in the nucleotide sequence set forth in SEQ ID NO:5 of the Sequence Listing or comprising at least a part thereof under stringent conditions, and that encodes a protein complex which is capable of enhancing tolerance to acetic acid.

5. The method of breeding cells according to claim 1, wherein the gene encoding the protein having a function of transporting a short chain fatty acid from the inside of cells to the outside of cells, is a DNA represented by the following (a) or (b):
(a) a DNA that comprises a nucleotide sequence consisting of nucleotide numbers 249 to 1025 in the nucleotide sequence set forth in SEQ ID NO: 8 of the Sequence Listing; or
(b) a DNA that comprises a nucleotide sequence which hybridizes with a probe comprising a nucleotide sequence consisting of nucleotide numbers 249 to 1025 in the nucleotide sequence set forth in SEQ ID NO: 8 of the Sequence Listing or comprising at least a part of said nucleotide sequence under stringent conditions, and that encodes a protein which is capable of enhancing tolerance to acetic acid.

6. The method of breeding cells according to any one of claims 1 to 5, wherein the short chain fatty acid is formic acid, acetic acid, propionic acid, butyric acid, isobutyric acid, or valeric acid.

7. Cells bred by the method according to any one of claims 1 to 5, wherein the cells are microbial cells.

8. The cells according to claim 7, wherein the microbial cells are cells of acetic acid bacteria, bacteria belonging to the genus Escherichia, or genus Bacillus.

9. A method for high cell-density culture that uses the cells according to claim 8.

10. The method for high cell-density culture according to claim 9, wherein the cells are cultured in the presence of a short chain fatty acid.

11. A method of preparing a fermentation broth comprising a short chain fatty acid, wherein the cells according to claim 8 are cultured under the conditions such that the cells produce a short chain fatty acid.


SEQUENCE LISTING (18 sequences)

SEQ ID NO: 1 - DNA, 2414 bp, Acetobacter aceti
gctcgcgtacccgggcgwtcctctagagtcatcaaccttggcccaggtggggatggcatacaggcggtggcggaaattacctcatctttcagtgtgccggttatatacgtaacggcgtatccagaacgtttgctaactggggaaaccatggagcccagttttgttattaccaagccgtttgacccccttacccttgctgttgcaacgtatcaggcagtaagcagcgcacgcacacaggccgtataagcaaaaaagcggcctccatttccagttctacaaaacggattatttttttccagcatggcgcatcctccccttcttcatcttcaggacattactctttcattaggagggaacccgctgctggatggcgccggttttgccgttgggcgtggtgagcgcctctgccttgtggggcgaaacggttcgggaaagtccaccctgctcaaaattgctgcgggtgttattcagccagattcggggtctgtgtttgtccagcccggtgcttccctgcgctatctgccgcaggagccggatttaagcgcttatgccacaacggcggattacgttgtgggccagattggagacccggatatggcatggcgcgccacgccattgctggatgctctgggcctgacaggtagggaaagcacgcaaaatctttcaggcggtgaaggtcggcgttgtgctattgctggtgtattggcggcggcccccgatgtgctgctgctggatgagcccaccaaccatctggatatgcctaccattgaatggttggagcgtgaactgctgagccttggcgccatggtaattatcagccatgataggcggctgctttccaccctttcacgttctgttgtgtggctggatcggggtgtaacccgcaggcttgatgaaggatttggaaggtttgaagcctggcgagaggaggttctggaacaggaagagcgtgatgcgcataaactggaccggaaaatcgcgcgggaagaagactggatgcgttatggcgtaacggcgcgccgcaaacgcaatgtacgccgtgtgcgggaactagcagatttgcgcacagcccgtaaggaggccattcgggcacccggcacccttaccttgaacacgcagctgcggccacatcgcaagctggtggctgtggccgaagatattagtaaggcatggggtgaaaagcaggttgttcgccatttggacctgcgcattttacgtggagaccggcttggtattgtgggggccaatggtgcaggcaaaaccacattgttgcggatgctaacagggctggaccaacccgatagtggcacaatctcacttggtccttcccttaatatggtcacgctggatcagcagcgacgtaccctgaacccggaacgcacactagccgataccttgacagaaggcggaggcgatatggtgcaggttggcacggaaaagcgccacgttgtggggtatatgaaagactttctgtttcggccagaacaggcacgcacacccgtaagtgccctttctggcggggagcgagggcggttaatgctggcatgcgcattggccaagccctccaacctgctggtgctggatgaacccaccaatgatctggatctggaaacactggatattttgcaagacatgctcgccagttgtgaaggcacagtgctgcttgtaagccatgatcgtgattttctggatcgggttgcaacatccgtcttggcgacagagggagatggcaactggatagaatatgctggcggatacagtgacatgctggctcagcggcaccagaaaccgttgacaacggcctctgtggtggaaaacgaacccacaaaacccaaagagacaactgctgcgcgtggcccgaccaaaaagctgagttataaggaccagtttgcgctggataatctgcccaaggaaatggaaaagctggaagcacaggctgccaactgcgtgaaaaactggcagatccagatttatatggaaaaaaccccgcgcagtttgagaaactttcggctgatttacagaagctcgaaacaaagctggcagaatctgaagaacgctggctggaactggaaatgaagcgagaagccctacaggccaactaaggcaacgctatttttcggtgaaccgcactcttgcaggcgggtgggtgcaatgcctatgttttggcatgctctgttttactggttctctttataagcgcacccttcctgctggcagtctggcattgcttgcctttctgagcgtggcacacattgcattcgcgcaggatadacccgccgctgcagtctccctatagtgagtcgtattacgcgttctaacgaatccatatgactwtgtagaccctctagagtcgacctgcaggcatgcaagcttyccctatagtgagtcgtattagagcttggcgtaatgcatga

SEQ ID NO: 2 - protein, 591 aa, Acetobacter aceti
MAHPPLLHLQDITLSLGGNPLLDGAGFAVGRGERLCLVGRNGSGKSTLLKIAAGVIQPDSGSVFVQPGASLRYLPQEPDLSAYATTADYVVGQIGDPDMAWRATPLLDALGLTGRESTQNLSGGEGRRCAIAGVLAAAPDVLLLDEPTNHLDMPTIEWLERELLSLGAMVIISHDRRLLSTLSRSVVWLDRGVTRRLDEGFGRFEAWREEVLEQEERDAHKLDRKIAREEDWMRYGVTARRKRNVRRVRELADLRTARKEAIRAPGTLTLNTQLRPHRKLVAVAEDISKAWGEKQVVRHLDLRILRGDRLGIVGANGAGKTTLLRMLTGLDQPDSGTISLGPSLNMVTLDQQRRTLNPERTLADTLTEGGGDMVQVGTEKRHVVGYMKDFLFRPEQARTPVSALSGGERGRLMLACALAKPSNLLVLDEPTNDLDLETLDILQDMLASCEGTVLLVSHDRDFLDRVATSVLATEGDGNWIEYAGGYSDMLAQRHQKPLTTASVVENEPTKPKETTAARGPTKKLSYKDQFALDNLPKEMEKLEAQAANCVKNWQIQIYMEKTPRSLRNFRLIYRSSKQSWQNLKNAGWNWK

SEQ ID NO: 3 - DNA, 2160 bp, Gluconacetobacter entanii
atcttgtggccaaggaattcatcgcagcccaggatcctgaaaatcccggcgtgctgatcctctcccgccttgccggggcggcaaagcagcttgaggccgccctgctggtcaacccgctggatcatgacggcatggccgatgcgctggaacgcgcgctggccatgtcgcccgaggaacggcgcgaacgctggcaggcatgctggaacagcattgccaaccgtacggcccttggctgggggctgtcgttcctgaacatccttgaaaacgcgaagcggcgttaagccccacaccagccttgcgcacggggtggttgagaatacataagtgggcatggcctcacctccccttcttctccttcaggatatcaccctgacccttggcggcgcgccgctgctcaatggcgcgggcttcggcgttggccctggcgagcgcgtctgccttgtcgggcgcaatggctgtggcaagtccaccctgctgcgcatcgcggcgggtgagatacaggccgatgacggcaccgtttttgtccagcccggcaccaccgtgcgctacctgccgcaggaacccgacctgtcgggctttgacaccacgctggattacgtccgcgcgggcatggggccgggcgacccggaataccgcgccgaactgctgctgaccgaactggggctgaacggcacggaagacccggccaccctgtcgggcggggaagcgcggcgctgcgcgctggcccgcgcccttgcgcccgaacccgacctgcttttgctggacgaacccaccaaccacctggacatgcccaccattgaatggctggaacgtgaactgctgtcgctgtcatcggccatggtcatcataagccatgaccgcaggctgctggaaacgctgtcgcgttcggtcgtgtggctggaccggggtgtcacccgcaggctggatcagggcttcgcccggttcgagacatggcgcgaggaagtgctggagcaggaagagcgcgacagccacaagctggaccgccagatcgcgcgtgaggaagactggatgcgttacggcgtgaccgcgcggcgcaagcgcaatgtccgccgcgtggctgaactggccgaactgcgcaatacccgtcgcaccgccataaggcagcccggcggcctgaagatggaagcccgcgaaagcgacctgtcgggcaagctggttgcggtggcagaagatatgtcacgcgcctatgaccctgcccacccggtggtcagccatctggacctgcgtgtcctgcgcggggaccggctggggatcgtgggggccaatggcgcgggcaagagcaccctgctgcgcctgctgacgggactggacaggccggattccggcaccatcaatatcggcagcgcgctcaatgtcgtcacactggaccagcagcgccgctcgcttgatcccgacaccacgctggcggatacgctgacgggcggcggcggggacatggtgcaggttggcaatgagaaacgccatgtcatcggctacatgaaggacttcctgttccgccccgaacaggcgcgtaccccggtgggcgtgctgtcggggggggagcgctggcggctcatgctggcctgcgcgctggcgcggccgtccaacctgctggtgctggacgagccgaccaacgaccttgaccttgaaacgctcgacctgctgcaggacatgctggccagctattccggcacggtgctgctggtcagccatgaccgtgacttcctcgaccgggtcgcctcctccatcctgatggcggaaggcggcggaaagtgggtggaatatgccggtggctacagcgacatgctggcccagcggcaggacgccacactggccgcccgcccccggcaggaccgcgcggaaaccacaccggccagaaccgatgtgaccccgtcctcctccccccggcagcccgcgcgcaagatgtcgtacaaggacaagcacgcgctggaacagctacccaagcagatggcggcgctggagacggaaatcgagcgcctgcgcgccatcctgtccgacgggggcctgtatgcgcgcgaccccgccacctttacggccgccaccacggcgctggaaaaggcagaggccgacctgacggcggcggaagaacggtggctggaactcgaaatgctgcgcgagacgcttcagtcttcctgaacg

SEQ ID NO: 4 - protein, 608 aa, Gluconacetobacter entanii
MASPPLLLLQDITLTLGGAPLLNGAGFGVGPGERVCLVGRNGCGKSTLLRIAAGEIQADDGTVFVQPGTTVRYLPQEPDLSGFDTTLDYVRAGMGPGDPEYRAELLLTELGLNGTEDPATLSGGEARRCALARALAPEPDLLLLDEPTNHLDMPTIEWLERELLSLSSAMVIISHDRRLLETLSRSVVWLDRGVTRRLDQGFARFETWREEVLEQEERDSHKLDRQIAREEDWMRYGVTARRKRNVRRVAELAELRNTRRTAIRQPGGLKMEARESDLSGKLVAVAEDMSRAYDPAHPVVSHLDLRVLRGDRLGIVGANGAGKSTLLRLLTGLDRPDSGTINIGSALNVVTLDQQRRSLDPDTTLADTLTGGGGDMVQVGNEKRHVIGYMKDFLFRPEQARTPVGVLSGGERWRLMLACALARPSNLLVLDEPTNDLDLETLDLLQDMLASYSGTVLLVSHDRDFLDRVASSILMAEGGGKWVEYAGGYSDMLAQRQDATLAARPRQDRAETTPARTDVTPSSSPRQPARKMSYKDKHALEQLPKQMAALETEIERLRAILSDGGLYARDPATFTAATTALEKAEADLTAAEERWLELEMLRETLQSS

SEQ ID NO: 5 - DNA, 3188 bp, Gluconacetobacter entanii
ccgggcgcgtggggacgaagtccggcccggactgatacgcgcgatcgcggatacgctggcgcgcgtgttgcccgcatggcccggcacgggcggcaacccgtccctgccgcgtgggcagatccatgccgacctgtttcccgacaatgtctttttccgtgacgggaaactgtcgggcatcattgatttctatttcgcctgcaccgactggtacgcctacgacctggccattaccgtcaatgcatggtgttttgatgacatgggccgtttcgtgcccacccgtgcgcgcgccatggtggaagcctaccgccatgtccgcccgctggaggatgcggaggacgcggccctggccacccttgccacgggtgcggccatccgtttcacgctgacccgcctgtatgactggatcaatacgcccccggatgcgctggtgacgcgcaaggacccgctggactatctggcgcgcatggaattcttcgcgtcccgcatggatgaaggtttcctgtgatggacgaggacatggcccccgtggaaaacgtggcacccgccaccgacatggtggaaatctggaccgatggcgggtgcaagcccaatcccggccccggtggctggggcgcattgctgtgctgtcgcgggcaggagcgtgaactgtcgggtggcgaggcggaaaccacaaacaaccgcatggaactgaccgccgcggccgaggcgctggaggcgctgaaacgtccctgccgtgtcgtgctgcacaccgacagcgaatatgtgcgcaatggcatcacgcggtggagcacgggctgggtgcggcgcaaatggcgcaatgcatccggtgatccggtggcgaacatggatctgtggcggcggctgctggatgtcagcgcgaagcacgagatcgagtggaaatgggtccgtggccattccggcgacgtgaataacgaacgcgtggaccagatggccacttcggcgcgtgacgcgctgggcattccctatcccaagcgtggaaaatgatgcgcgcgccccctgccatccgtctggaaggggtggggctggatttcggggccgcgccgcttttccgtaatctggacctgtgcatcggggccgggaccatgaccgtgctgctgggtgccagcggtgttggcaagacatcgctgctgcgcatgctgggcggcttggtcgcccctgattactggcgtgtcgtggccggggacgggctgccccttgccggacgggtcgcatggatggggcagcaggatctgctgctgccatgggcgactgccatggacaatgtaatgctgggcgcgcgcctgcgtggcgaccggctggaccgcgacagggcagggtgcctgctggattgtgtcgggctgtcatcgcacgcggcggcccttccggccacgctgtcggggggcatgcggcagcgtgtggcgctggcgctggtattgtatgaagaccgtccggtcgtgctgatggatgaacctttttccgcactggacagtgtgacacgggcacgcatgcaggatctggccggccggatgctggcgggccggaccgtggtgctgattacccatgacccgctggaagcctgccgtctggcggatcacatgctgctcatggccgggcatcccgcacggctggacggcattgccgtcccggcggggaccgtgccgcgcgcggtggatgacgcgggcgtgcttgcggcgcagtccgcgctgctgcggcggatgatgcaatgaccggcaaggccacggcacggggcatgcgcctgctgcgcccgcttgtcacgctggccggtctggtggccgtgtggggcgcgcttgcgcgctgggggcatgtgccgccctatatgctgcccggccccgatgcggtggcgcgtgcgctgtggacgcagcgcgcgcaactggccccagccgccctgaccacgctggaggagacggttctgggccttgtgctgggaatcggggcggggggcgcgctggccatcggcatggcggtctgcgcgccgctgcggcggtgggtcatgcccatggtgctgctcagccaggcggtgccggttttcgcgctggcgccgctgctggtgctgtggttcggattcggcatggcgtccaaggtggtcatggcggtgctggtcattttctttcccgtcacgtcggcgcttggcgatggcctgcgccagaccgagccggggtggatggaccttgcccgcaccatgggggccacgcggtggcgggtgctggtgcatgtgcgcctgcccgcggccctgccgtcctttgcctcgggcgtgcgcatggccaccgccatcgccccgatcggggccgtcgtgggggagtgggtcggggcgtcgtccggcctctggttcctgatgcagacggccaatacccgattccagacggatctcatgtttgccgctctcgcggtgctggcggtcatgaccgtgctgctgtggtggggcgtggaccggttgctggcgcgtgcgctgtactggctgccccggcatgctgatgttgactgaactgttgtaacacgtcaccaccaccgctaatcgcgcaacccaatgcaggatggatgcccaatgacacgccccatcattcttgacctgacacccggcgcgcagggaatggcgaccctgctggcggcgctgcaggtgcctgaccacgtgcgcccggtgctggtgctgttcagcggcagggccgccgaagtcgaagtggcgctgacccatgcgcgtgacctgctgcaccggtacgggctggatgatgtctccgtgtgcgcgggctgtcccggcccgatggtgcaggccggggacgtggggcatgacctgcccgcggggcaggatgggctgggcgcgctgcatctggtgcgggcggtgcgggcctgttcggcggacagcgtgagcatatgctgttccggcccgctgaccacgctggccgtggccctggtgcaggcgcccgacatggcttcgcacctgcatggggtgattgtgaatggcggcgcgtttttcgtgcatggcgatgccaccagcgtggccgaacgcaatatcgcggccgacccggaagccgcggctgtggtgctggcggcgggcgtgccggtgaccattgtgccgctggactgcgccgcgcgcctgacggcggatgcggtgtggatggaacagcttgagccgatgggccgcgtcccggcttccgtcgcgggcagggtgcatg

SEQ ID NO: 6 - protein, 241 aa, Gluconacetobacter entanii
MMRAPPAIRLEGVGLDFGAAPLFRNLDLCIGAGTMTVLLGASGVGKTSLLRMLGGLVAPDYWRVVAGDGLPLAGRVAWMGQQDLLLPWATAMDNVMLGARLRGDRLDRDRAGCLLDCVGLSSHAAALPATLSGGMRQRVALALVLYEDRPVVLMDEPFSALDSVTRARMQDLAGRMLAGRTVVLITHDPLEACRLADHMLLMAGHPARLDGIAVPAGTVPRAVDDAGVLAAQSALLRRMMQ

SEQ ID NO: 7 - protein, 259 aa, Gluconacetobacter entanii
MTGKATARGMRLLRPLVTLAGLVAVWGALARWGHVPPYMLPGPDAVARALWTQRAQLAPAALTTLEETVLGLVLGIGAGGALAIGMAVCAPLRRWVMPMVLLSQAVPVFALAPLLVLWFGFGMASKVVMAVLVIFFPVTSALGDGLRQTEPGWMDLARTMGATRWRVLVHVRLPAALPSFASGVRMATAIAPIGAVVGEWVGASSGLWFLMQTANTRFQTDLMFAALAVLAVMTVLLWWGVDRLLARALYWLPRHADVD

SEQ ID NO: 8 - DNA, 1260 bp, Gluconacetobacter entanii
ggccatatcgtgatccgcacccgcacggagaccataaccggggaccggggcgtatatgtgcccgatacgggcattgcccgtctggttggcaacgtgcatatcacccgtggggaaaatcaggtcagcggcacgtcggccatcgtgaacatgcataccgatatcgcgacgctgaccgataaccccggctcacgtgtcagcggcctggtcattccgaaccaggctggcaaaggtgacaagggatcagcaagatgaacacgacatccccggcggaccaggccaccaccgaacagccgatccccgcagccgaagcggggctgatcgccagcggcatcggcaagagctacaagaaacggcaggtcgtgcgcgatgtctcgctgcaggtccagcgtggcgaggccgtggccctgctggggccgaacggcgcgggcaagaccaccagcttctacatgatcgtgggactggtgcggcccgacatgggcacgatcacgctggatggcgcggatatcacccagttgcccatgtaccgccgcgcccgcatgggcattggctacctgccgcaggaatcaagcattttccgtggcctgaatgttgaacagaacatcatggcggcgctggagatcgtggaacccgaccgggaccggcggcagacgatgcttgacgggctgctgggggaattcggcattacccggctgcggcattcatcctccctcgccctgtcgggcggggagcggcggcggctggaaatcgcccgcgcgctggccagccagccgcattatatcctgctggacgaaccgctggccggtatcgaccccattgcggtgggcgagatccgtgaccttgtcgcccacctgaaggatcgtggcatcggggtgctgattaccgaccacaacgtgcgcgagacgctggaagtgatcgaccgggcctacatcatgcacagcgggcaggtgctgaccgagggacggcccgaggaaatcgtggcgaacgaagacgtgcgccgtgtctatctgggtgaaaaattcacgctgtaggcccgtacgcgcgcccctgtgtgacagggtgcatgtgcaaggggaaggccatggaaaaacctgtggtcttttatgcaaggaaggcatggacagcaacggtgcaaccatgcccaaatgtacccgttcgggaccatgtgcccgacaggcacaacgcaaccgccaccagcacgcagccagcatgggagcaggatcgtcccccttatttcatgaccattgactgaaacgaccgtat

SEQ ID NO: 9 - protein, 259 aa, Gluconacetobacter entanii
MNTTSPADQATTEQPIPAAEAGLIASGIGKSYKKRQVVRDVSLQVQRGEAVALLGPNGAGKTTSFYMIVGLVRPDMGTITLDGADITQLPMYRRARMGIGYLPQESSIFRGLNVEQNIMAALEIVEPDRDRRQTMLDGLLGEFGITRLRHSSSLALSGGERRRLEIARALASQPHYILLDEPLAGIDPIAVGEIRDLVAHLKDRGIGVLITDHNVRETLEVIDRAYIMHSGQVLTEGRPEEIVANEDVRRVYLGEKFTL

SEQ ID NO: 10 - DNA, 30 bp, Artificial
  Description of Artificial sequence: synthetic oligonucleotide
cgctgacgtcgtgggccgtgccagaggccc

SEQ ID NO: 11 - DNA, 30 bp, Artificial
  Description of Artificial sequence: synthetic oligonucleotide
ggccaagacgtctgcagcatggggcgtcac

SEQ ID NO: 12 - DNA, 5734 bp, Gluconacetobacter entanii (Acetobacter altoacetigenes MH-24)
catggggcgtcacccccagcggccagcttggctacctgatggacagggcgggccttctgcaagccctcggccactgccatctgccgggatatgaggccaaatacgaaccgaaggaaaagcgcaccttctgctaccccacccagaacgccagcggctgggctgtgcagccatgatcgccaacccctccctcttcctgagcaattcggaagagcgatttccgccgactgaacacgtcgaaaatggcagttttccaccgaaaaaaggaaaggaccataggaaaggattaatatcttatttttatctaggggtttgccgatccgcgattttcgctgggaaaccgccaaaaatggcttgccattaggtcgcaccacatgcgaccataaagtcgcacagtgtgcgacctattcggcccatatacagaggttccccacatgcggaatgtcacccgtctcaagacccgcaaagaccggctccgcgaggaccaagccgacctgttgaagcaagcccttctgcccttcgcagaggacgatggaccgatgcgggatgcggtcggacggctctacgtccagatcaagaacctcaccaccccagaccccggaaccacggagccgttcgtcatgatccgtcccgcccagaatcgcgccgtcaccctctggctgctgaagaacagtaagcggcccatgaaggccgtggacgtatggacgctgctgttcgaccacctgtttccccataccggccagatcatgctgacccgtgaggaaatcgcggaaaaagtcggtatccgggtcaacgaagttacagccgtcatgaacgagctggtgagcttcggcgcgattttctccgagcgcgagaaggtggccggaatgcgcgggccgggcctcgcccgctactacatgaaccggcatgtggccgaggtcggcagccgcgccacgcaggaagaacttgccctaatcccacgccccggcgccaagctggcagtcgtgcagggtggcaaggcttaacccatgaaggtttcggaactggacgtgttcgacagcgccaaggcggcacaagacccgttggtgcgggaagaactgctgcaagcagcgcaggcggacggccacggccccgccctcgctcatgcccgttccgtcatagccaaggcgcgggccgggcaggacgccaaggcttaacggccccgccctctcccgcctcgatcccggcgggcctgtagcatctcctgatgctccttggcgtttttggcccgctgctcggcccgctctttctcggccgctgcggctcttaggcgctcttcggccagccgcatccgctcgtccatctgacgtttccgatctgcctcggcatccttggcggctcctgccttcagccctttgctgaaagccatccacttattggcggttttctcggctttctgctgtatcggcggggtcagccggtcaaatgcctgggccaccctctcgaagccctcacgcatggcgttgacggcctgcgccagtttagccagggcgaaatctatcacctcggcccgctgggcgttctcggcccggatacgccggttgtggttgccggtcggggtctggtggcccttccgttccagagccaccacattcggccccatgtgccgctctggaacgcggtctagcccctgctccgcattgctccggtgatctatccgggcctcttgcccagcccgctctagcgcggcattggcaaggcccgcccatagctgccggatttccttcacctcgtcggcggccttccccagtcccatgccctgccgcttcttgtcggacagttcgatggttgatttgtctccaaaggacagcttgccatcggccccccgctccaccgtgcgggtggtggtcatgatgtgcgcgtgatgattccggtcgtcgccctcgtcacccggaagatgcacggccacgtccacggccaccccgtaccgctggaccaactcacgcgcgaaactgtccgccagttcggcccgctgctcgctggtgagttcatgagggagggccacaacccattccctcccggtgcgggcgtccttgcgtttctctgatcgctccgcgtcattccacaattccgaacggtcagcggtgccaccccccggaatgaaaattgccttatgggcaacgctattctgcctggggctgtatttgtgttcgtgcccgtcaacctcgttggtcaaatcctcgccagcacgatacgcagccgcagccacaacggaacgccctgcgctccggctgatcggtttcgtttctgcgcgatagattgccacggatcgagcgcctaccttttggagttaaacggggggttcaggggggcgaagccaccatgacgcaggacttgcacttgtgcaagtcgtaactgcgcccttaatacctgacggcatcaagggatatgtggtattcgtttgaaacggaacggctccacggtgaggatgatatgagcgatattgcgaaagagattgagaacgccaaaaggatcatagctgaacagaaaaagcgcatcaaagatgcccagaaggaagcagctaaagcggaatcaaagttgagggaccgtcagaactacatcttgggcggcgcactggtaaaacttgccgaaacagatgaacgggccgtccgcactattgaaacacttttgaagctggtggatcgtccatcagaccggaaggcgtttgaggtgttttcccgtctcccatccctctccctgcccacgcagccagcaccggacaccggccatgagtgaggcactggaagaagatccgtttgaactgttcaaaagggtcgaaaaaagcctgtccacggccaccgccagcatggagcggctggccgccgaacaagatgccaggtgcaagaccatttcagacgccgccggaaaagcctctaaattggccgaggaagccggtgacaccttcacagcatccaagaggcgtctgatgatctggacggccctctgcgcggctctgctggtctgtggcgggtggttggcgggttattggctgggacaccgtgacggttgggcctctggcacggcccacgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccattcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 30 bp, Artificial
  Description of Artificial Sequence: synthetic oligonucleotide
tgaacatccttgaattcgcgaagcggcgtt

SEQ ID NO: 14 - DNA, 27 bp, Artificial
  Description of Artificial Sequence: synthetic oligonucleotide
acggatccaggcgtccggcctgatcat

SEQ ID NO: 15 - DNA, 22 bp, Artificial
  Description of Artificial Sequence: synthetic oligonucleotide
aagcacgagatcgagtggaaat

SEQ ID NO: 16 - DNA, 22 bp, Artificial
  Description of Artificial Sequence: synthetic oligonucleotide
gggtgtcaggtcaagaatgatg

SEQ ID NO: 17 - DNA, 20 bp, Artificial
  Description of Artificial sequence: synthetic oligonucleotide
gccatcgtgaacatgcatac

SEQ ID NO: 18 - DNA, 22 bp, Artificial
  Description of Artificial sequence: synthetic oligonucleotide
ccttccttgcataaaagaccac